(19) United States
(12) Patent Application Publication
(10) Pub. No.: US 2024/0174669 A1
(43) Pub. Date: May 30, 2024

(54) CRYSTALLINE FORMS OF A PARP1 INHIBITOR

(71) Applicant: XinThera, Inc., Foster City, CA (US)

(72) Inventors: Jayachandra P. REDDY, West Palm Beach, FL (US); Daniel John GANLEY, Syracuse, NY (US); Robert L. HOFFMAN, San Diego, CA (US)

(21) Appl. No.: 18/481,723

(22) Filed: Oct. 5, 2023

Related U.S. Application Data
(60) Provisional application No. 63/378,579, filed on Oct. 6, 2022.

(51) Int. Cl. C07D 471/04

(52) U.S. Cl. CPC C07D 471/04

(57) ABSTRACT

Described herein are crystalline forms of 5-(4-((7-cyclopropyl-6-oxo-5,6-dihydro-1,5-naphthyridin-3-yl)methylipiperazin-1-yl)-N-methylpicolinamide (compound 1), or a pharmaceutically acceptable salt of solvate thereof.

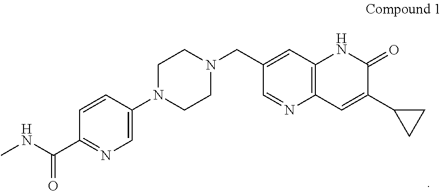 [SMILES: CNC(=O)c1ccc(N2CCN(Cc3cnc4cc(C5CC5)c(=O)[nH]c4c3)CC2)cn1]

### CROSS-REFERENCE TO RELATED APPLICATIONS

This application claims the benefit under 35 U.S.C. § 119(e) to U.S. Provisional Application No. 63/378,579, filed Oct. 6, 2022, which is hereby incorporated by reference in its entirety.

### BACKGROUND

Poly(ADP-ribose)polymerase (PARP) or poly(ADP-ribose)synthase (PARS) has an essential role in facilitating DNA repair, controlling RNA transcription, mediating cell death, and regulating immune response. These actions make PARP inhibitors targets for a broad spectrum of disorders. PARP inhibitors have demonstrated efficacy in numerous models of disease, particularly in models of ischemia reperfusion injury, inflammatory disease, degenerative diseases, protection from adverse effects of cytotoxic compounds, and the potentiation of cytotoxic cancer therapy. PARP has also been indicated in retroviral infection and thus inhibitors may have use in antiretroviral therapy. PARP inhibitors have been efficacious in preventing ischemia reperfusion injury in models of myocardial infarction, stroke, other neural trauma, organ transplantation, as well as reperfusion of the eye, kidney, gut, and skeletal muscle. Inhibitors have been efficacious in inflammatory diseases such as arthritis, gout, inflammatory bowel disease, CNS inflammation such as MS and allergic encephalitis, sepsis, septic shock, hemorrhagic shock, pulmonary fibrosis, and uveitis. PARP inhibitors have also shown benefit in several models of degenerative disease including diabetes (as well as complications) and Parkinson's disease. PARP inhibitors can ameliorate the liver toxicity following acetaminophen overdose, cardiac and kidney toxicities from doxorubicin and platinum based antineoplastic agents, as well as skin damage secondary to sulfur mustards. In various cancer models, PARP inhibitors have been shown to potentiate radiation and chemotherapy by increasing cell death of cancer cells, limiting tumor growth, decreasing metastasis, and prolonging the survival of tumor-bearing animals.

PARP1 and PARP2 are the most extensively studied PARPs for their role in DNA damage repair. PARP1 is activated by DNA damage breaks and functions to catalyze the addition of poly (ADP-ribose) (PAR) chains to target proteins. This post-translational modification, known as PARylation, mediates the recruitment of additional DNA repair factors to DNA lesions.

Following completion of this recruitment role, PARP auto-PARylation triggers the release of bound PARP from DNA to allow access to other DNA repair proteins to complete repair. Thus, the binding of PARP to damaged sites, its catalytic activity, and its eventual release from DNA are all important steps for a cancer cell to respond to DNA damage caused by chemotherapeutic agents and radiation therapy.

Inhibition of PARP family enzymes has been exploited as a strategy to selectively kill cancer cells by inactivating complementary DNA repair pathways. A number of pre-clinical and clinical studies have demonstrated that tumor cells bearing deleterious alterations of BRCA1 or BRCA2, key tumor suppressor proteins involved in double-strand DNA break (DSB) repair by homologous recombination (HR), are selectively sensitive to small molecule inhibitors of the PARP family of DNA repair enzymes. Such tumors have deficient homologous recombination repair (HRR) pathways and are dependent on PARP enzymes function for survival. Although PARP inhibitor therapy has predominantly targeted SRCA-mutated cancers, PARP inhibitors have been tested clinically in non-SRCA-mutant tumors, those which exhibit homologous recombination deficiency (HRD).

It is believed that PARP inhibitors having improved selectivity for PARP1 may possess improved efficacy and reduced toxicity compared to other clinical PARP1/2 inhibitors. It is believed also that selective strong inhibition of PARP1 would lead to trapping of PARP1 on DNA, resulting in DNA double strand breaks (DSBs) through collapse of replication forks in S-phase. It is believed also that PARP1—DNA trapping is an effective mechanism for selectively killing tumor cells having HRD. An unmet medical need therefore exists for effective and safe PARP inhibitors. Especially PARP inhibitors having selectivity for PARP1.

### SUMMARY

Disclosed herein is a crystalline form of 5-(4-((7-cyclopropyl-6-oxo-5,6-dihydro-1,5-naphthyridin-3-yl)methylipiperazin-1-yl)-N-methylpicolinamide (compound 1):

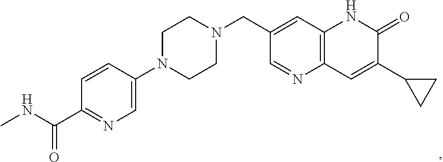 [SMILES: CNC(=O)c1ccc(N2CCN(Cc3cnc4cc(C5CC5)c(=O)[nH]c4c3)CC2)cn1]

or a pharmaceutically acceptable salt or solvate thereof.

Also disclosed herein is a crystalline form of freebase 5-(4-((7-cyclopropyl-6-oxo-5,6-dihydro-1,5-naphthyridin-3-yl)methyl)piperazin-1-yl)-N-methylpicolinamide (compound 1):

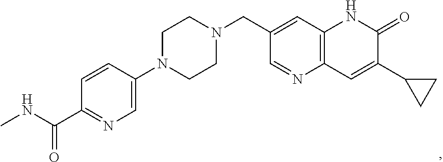 [SMILES: CNC(=O)c1ccc(N2CCN(Cc3cnc4cc(C5CC5)c(=O)[nH]c4c3)CC2)cn1]

or a pharmaceutically acceptable solvate thereof.

Also disclosed herein is a crystalline form of freebase 5-(4-((7-cyclopropyl-6-oxo-5,6-dihydro-1,5-naphthyridin-3-yl)methyl)piperazin-1-yl)-N-methylpicolinamide (compound 1):

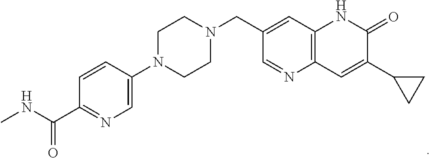 [SMILES: CNC(=O)c1ccc(N2CCN(Cc3cnc4cc(C5CC5)c(=O)[nH]c4c3)CC2)cn1]

In some embodiments of a crystalline form, the crystalline form is selected from group consisting of Form I of compound 1 and Form II of compound 1, or any combinations thereof.

In some embodiments of a crystalline form, the crystalline form is selected from the group consisting of freebase Form I of compound 1, and freehase Form H of compound I, or any combinations thereof.

In some embodiments of a crystalline form, the crystalline compound 1 is Form II characterized as having at least one of the following properties:
(a) an X-ray powder diffraction (XRPD) pattern substantially the same as shown in FIG. 2;
(b) an X-ray powder diffraction (XRPD) pattern with characteristic peaks at 20.88±0.2° 2θ, 21.37±0.2° 2θ, and ±25.31 0.2° 2θ.
(c) a DSC thermogram with an endotherm having an onset temperature at about 265.5° C. and a peak temperature at about 266.5° C.; or
(d) combinations thereof.

In some embodiments of a crystalline form, the crystalline form has an X-ray powder diffraction (XRPD) pattern substantially the same as shown in FIG. 2.

In some embodiments of a crystalline form, crystalline compound 1, Form II has an X-ray powder diffraction (XRPD) pattern with characteristic peaks found in Table 2.

In some embodiments of a crystalline form, crystalline compound 1, Form II has an X-ray powder diffraction (XRPD) pattern with characteristic peaks at 20.88±0.2° 2θ, 21.37±0.2° 2θ, and ±25.310.2° 2θ.

In some embodiments of a crystalline form, the X-ray powder diffraction pattern further comprises a peak at 10.96±0.2° 2θ.

In some embodiments of a crystalline form, the X-ray powder diffraction pattern further comprises a peak at 16.57±0.2° 2θ.

In some embodiments of a crystalline form, the X-ray powder diffraction pattern further comprises a peak at 17.26±0.2° 2θ.

In some embodiments of a crystalline form, the X-ray powder diffraction pattern further comprises a peak at 17.71±0.2° 2θ.

In some embodiments of a crystalline form, the X-ray powder diffraction pattern further comprises a peak at 17.93±0.2° 2θ.

In some embodiments of a crystalline form, the X-ray powder diffraction pattern further comprises a peak at 19.54±0.2° 2θ.

In some embodiments of a crystalline form, the X-ray powder diffraction pattern further comprises a peak at 23.25±0.2° 2θ.

In some embodiments of a crystalline form, the X-ray powder diffraction pattern further comprises a peak at 26.27±0.2° 2θ.

In some embodiments of a crystalline form, crystalline compound 1, Form II has an X-ray powder diffraction (XRPD) pattern with characteristic peaks at 10.96±0.2° 2θ, 16.57±0.2° 2θ, 17.26±0.2° 2θ, 17.71±0.2° 2θ, 17.93±0.2° 2θ, 19.54±0.2° 2θ, 20.88±0.2° 2θ, 21.37±0.2° 2θ, 23.25±0.2° 2θ, 25.31 0.2° 2θ, and 26.27±0.2° 2θ.

In some embodiments of a crystalline form, crystalline compound 1, Form II has an X-ray powder diffraction (XRPD) pattern with at least five characteristic peaks selected from 10.96±0.2° 2θ, 16.57±0.2° 2θ, 17.26±0.2° 2θ, 17.71±0.2° 2θ, 17.93±0.2° 2θ, 19.54±0.2° 2θ, 20.88±0.2° 2θ, 21.37±0.2° 2θ, 23.25±0.2° 2θ, 25.31 0.2° 2θ, and 26.27±0.2° 2θ.

In some embodiments of a crystalline form, crystalline compound 1, Form II has an X-ray powder diffraction (XRPD) pattern with at least six characteristic peaks selected from 10.96±0.2° 2θ, 16.57±0.2° 2θ, 17.26±0.2° 2θ, 17.71±0.2° 2θ, 17.93±0.2° 2θ, 19.54±0.2° 2θ, 20.88±0.2° 2θ, 21.37±0.2° 2θ, 23.25±0.2° 2θ, 25.31 0.2° 2θ, and 26.27±0.2° 2θ.

In some embodiments of a crystalline form, crystalline compound 1, Form II has an X-ray powder diffraction (XRPD) pattern with at least seven characteristic peaks selected from 10.96±0.2° 2θ, 16.57±0.2° 2θ, 17.26±0.2° 2θ, 17.71±0.2° 2θ, 17.93±0.2° 2θ, 19.54±0.2° 2θ, 20.88±0.2° 2θ, 21.37±0.2° 2θ, 23.25±0.2° 2θ, 25.31 0.2° 2θ, and 26.27±0.2° 2θ.

In some embodiments of a crystalline form, crystalline compound 1, Form II has an X-ray powder diffraction (XRPD) pattern with at least eight characteristic peaks selected from 10.96±0.2° 2θ, 16.57±0.2° 2θ, 17.26±0.2° 2θ, 17.71±0.2° 2θ, 17.93±0.2° 2θ, 19.54±0.2° 2θ, 20.88±0.2° 2θ, 21.37±0.2° 2θ, 23.25±0.2° 2θ, 25.31 0.2° 2θ, and 26.27±0.2° 2θ.

In some embodiments of a crystalline form, crystalline compound 1, Form II has a DSC thermogram with an endotherm having an onset temperature at about 265.5° C. and a peak temperature at about 266.5° C.

In some embodiments of a crystalline form, crystalline compound 1, Form II is anhydrous.

In some embodiments of a crystalline form, crystalline compound 1, Form II is physically and chemically stable.

In some embodiments of a crystalline form, crystalline compound 1, Form II is physically and chemically stable at both 60° C. (capped) and 40° C./75% RH (open) after 7 days.

In some embodiments of a crystalline form, the crystalline compound 1 is Form I characterized as having at least one of the following properties:
(a) an X-ray powder diffraction (XRPD) pattern substantially the same as shown in FIG. 1;
(b) an X-ray powder diffraction (XRPD) pattern with characteristic peaks at 8.11±0.2° 2θ, 16.80±0.2° 2θ, 19.35±0.2° 2θ, 20.44±0.2° 2θ, and 24.46±0.2° 2θ.
(c) a DSC thermogram with an endotherm having a peak temperature at about 255° C.;
(d) a DSC thermogram with an endotherm having a peak temperature at about 258° C.;
(e) a DSC thermogram with an endotherm having a peak temperature at about 266° C.;
(f) a thermogravimetric analysis (TGA) thermogram comprising a loss in mass of about 1.4% over a temperature range of about 70° C. to about 255° C.; or
(g) combinations thereof.

In some embodiments of a crystalline form, crystalline compound 1, Form I has an X-ray powder diffraction (XRPD) pattern substantially the same as shown in FIG. 1.

In some embodiments of a crystalline form, crystalline compound 1, Form I has an X-ray powder diffraction (XRPD) pattern with characteristic peaks found in Table 1.

In some embodiments of a crystalline form, crystalline compound 1, Form I has an X-ray powder diffraction (XRPD) pattern with characteristic peaks at 8.11±0.2° 2θ, 16.80±0.2° 2θ, 19.35±0.2° 2θ, 20.44±0.2° 2θ, and 24.46±0.2° 2θ.

In some embodiments of a crystalline form, crystalline compound 1, Form I has an X-ray powder diffraction (XRPD) pattern with at least three characteristic peaks selected from 8.11±0.2° 2θ, 16.80±0.2° 2θ, 19.35±0.2° 2θ, 20.44±0.2° 2θ, and 24.46±0.2° 2θ.

In some embodiments of a crystalline form, crystalline compound 1, Form I has an X-ray powder diffraction (XRPD) pattern with at least four characteristic peaks selected from 8.11±0.2° 2θ, 16.80±0.2° 2θ, 19.35±0.2° 2θ, 20.44±0.2° 2θ, and 24.46±0.2° 2θ.

In some embodiments of a crystalline form, the X-ray powder diffraction pattern further comprises at least one peak selected from 10.10±0.2° 2θ, 12.14±0.2° 2θ, 22.25±0.2° 2θ, and 25.22±0.2° 2θ.

In some embodiments of a crystalline form, the X-ray powder diffraction pattern further comprises at least two peaks selected from 10.10±0.2° 2θ, 12.14±0.2° 2θ, 22.25±0.2° 2θ, and 25.22±0.2° 2θ.

In some embodiments of a crystalline form, the X-ray powder diffraction pattern further comprises at least three peaks selected from 10.10±0.2° 2θ, 12.14±0.2° 2θ, 22.25±0.2° 2θ, and 25.22±0.2° 2θ.

In some embodiments of a crystalline form, the X-ray powder diffraction pattern further comprises peaks at 10.10±0.2° 2θ, 12.14±0.2° 2θ, 22.25±0.2° 2θ, and 25.22±0.2° 2θ.

In some embodiments of a crystalline form, crystalline compound 1, Form I has an X-ray powder diffraction (XRPD) pattern with characteristic peaks at 8.11±0.2° 2θ, 10.10±0.2° 2θ, 12.14±0.2° 2θ, 16.80±0.2° 2θ, 19.35±0.2° 2θ, 20.44±0.2° 2θ, 22.25±0.2° 2θ, 24.46±0.2° 2θ, and 25.22±0.2° 2θ.

In some embodiments of a crystalline form, crystalline compound 1, Form I has an X-ray powder diffraction (XRPD) pattern with at least five characteristic peaks selected from 8.11±0.2° 2θ, 10.10±0.2° 2θ, 12.14±0.2° 2θ, 16.80±0.2° 2θ, 19.35±0.2° 2θ, 20.44±0.2° 2θ, 22.25±0.2° 2θ, 24.46±0.2° 2θ, and 25.22±0.2° 2θ.

In some embodiments of a crystalline form, crystalline compound 1, Form I has an X-ray powder diffraction (XRPD) pattern with at least six characteristic peaks selected from 8.11±0.2° 2θ, 10.10±0.2° 2θ, 12.14±0.2° 2θ, 16.80±0.2° 2θ, 19.35±0.2° 2θ, 20.44±0.2° 2θ, 22.25±0.2° 2θ, 24.46±0.2° 2θ, and 25.22±0.2° 2θ.

In some embodiments of a crystalline form, crystalline compound 1, Form I has an X-ray powder diffraction (XRPD) pattern with at least seven characteristic peaks selected from 8.11±0.2° 2θ, 10.10±0.2° 2θ, 12.14±0.2° 2θ, 16.80±0.2° 2θ, 19.35±0.2° 2θ, 20.44±0.2° 2θ, 22.25±0.2° 2θ, 24.46±0.2° 2θ, and 25.22±0.2° 2θ.

In some embodiments of a crystalline form, crystalline compound 1, Form I has an X-ray powder diffraction (XRPD) pattern with at least eight characteristic peaks selected from 8.11±0.2° 2θ, 10.10±0.2° 2θ, 12.14±0.2° 2θ, 16.80±0.2° 2θ, 19.35±0.2° 2θ, 20.44±0.2° 2θ, 22.25±0.2° 2θ, 24.46±0.2° 2θ, and 25.22±0.2° 2θ.

In some embodiments of a crystalline form, the X-ray powder diffraction pattern further comprises at least one peak selected from 17.90±0.2° 2θ, 24.36±0.2° 2θ, and 28.18±0.2° 2θ.

In some embodiments of a crystalline form, crystalline compound 1, Form I has a DSC thermogram with an endotherm having a peak temperature at about 255° C.

In some embodiments of a crystalline form, crystalline compound 1, Form I has a DSC thermogram with an endotherm having a peak temperature at about 258° C.

In some embodiments of a crystalline form, crystalline compound 1, Form I has a DSC thermogram with an endotherm having a peak temperature at about 266° C.

In some embodiments of a crystalline form, crystalline compound 1, Form I has a thermogravimetric analysis (TGA) thermogram comprising a loss in mass of about 1.4% over a temperature range of about 70° C. to about 255° C.

In some embodiments of a crystalline form, crystalline compound 1, Form I is anhydrous.

Also disclosed herein is a pharmaceutical composition comprising a crystalline form disclosed herein, and a pharmaceutically acceptable excipient.

Also disclosed herein is a method of treating cancer in a subject in need thereof, the method comprising administering a crystalline form disclosed herein.

In some embodiments, the cancer is breast cancer, ovarian cancer, pancreatic cancer, prostate cancer, a hematological cancer, gastrointestinal cancer, or lung cancer.

Also disclosed herein is a method of treating a cancer comprising a BRCA1 and/or a BRCA2 mutation in a subject in need thereof, the method comprising administering a crystalline form disclosed herein.

In some embodiments, the cancer is bladder cancer, brain & CNS cancers, breast cancer, cervical cancer, colorectal cancer, esophagus cancer, Hodgkin lymphoma, non-Hodgkin lymphoma, kidney cancer, leukemia, lung cancer, melanoma, myeloma, oral cavity cancer, ovarian cancer, pancreatic cancer, prostate cancer, skin cancer, stomach cancer, thyroid cancer, or uterus cancer.

### INCORPORATION BY REFERENCE

All publications, patents, and patent applications mentioned in this specification are herein incorporated by reference to the extent applicable and relevant and to the same extent as if each individual publication, patent, or patent application was specifically and individually indicated to be incorporated by reference.

### BRIEF DESCRIPTION OF THE FIGURES

FIG. 1 shows the X-ray powder diffraction (XRPD) pattern for crystalline compound 1, Form I.

FIG. 2 shows the X-ray powder diffraction (XRPD) pattern for crystalline compound 1, Form II.

FIG. 3 shows the X-ray powder diffraction (XRPD) overlay pattern for crystalline compound 1, Form I and II and pattern III.

### DETAILED DESCRIPTION

### Definitions

In the following description, certain specific details are set forth in order to provide a thorough understanding of various embodiments. However, one skilled in the art will understand that the invention may be practiced without these details. In other instances, well-known structures have not been shown or described in detail to avoid unnecessarily obscuring descriptions of the embodiments. Unless the context requires otherwise, throughout the specification and claims which follow, the word “comprise” and variations thereof, such as, “comprises” and “comprising” are to be construed in an open, inclusive sense, that is, as “including, but not limited to.” Further, headings provided herein are for convenience only and do not interpret the scope or meaning of the claimed invention.

Reference throughout this specification to “some embodiments” or “an embodiment” means that a particular feature, structure, or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

Reference to “about” a value or parameter herein includes (and describes) embodiments that are directed to that value or parameter per se. In certain embodiments, the term “about” includes the indicated amount±10%. In other embodiments, the term “about” includes the indicated amount±5%. In certain other embodiments, the term “about” includes the indicated amount±1%.

An “effective amount” or “therapeutically effective amount” refers to an amount of a compound administered to a mammalian subject, either as a single dose or as part of a series of doses, which is effective to produce a desired therapeutic effect.

“Treatment” of an individual (e.g. a mammal, such as a human) or a cell is any type of intervention used in an attempt to alter the natural course of the individual or cell. In some embodiments, treatment includes administration of a pharmaceutical composition, subsequent to the initiation of a pathologic event or contact with an etiologic agent and includes stabilization of the condition (e.g., condition does not worsen) or alleviation of the condition.

“Synergy” or “synergize” refers to an effect of a combination that is greater than additive of the effects of each component alone at the same doses.

As used herein, a “disease or disorder associated with PARP” or, alternatively, “a PARP-mediated disease or disorder” means any disease or other deleterious condition in which PARP, or a mutant thereof, is known or suspected to play a role.

As used herein, a “disease or disorder associated with PARP1” or, alternatively, “a PARP1-mediated disease or disorder” means any disease or other deleterious condition in which PARP, or a mutant thereof, is known or suspected to play a role.

The term “substantially the same as” as used herein, refers to a powder X-ray diffraction pattern. DSC thermogram, or TGA pattern that is identical or non-identical to those depicted herein, but that falls within the limits of experimental error, when considered by one of ordinary skill in the art.

The term “substantially similar to” as used herein, refers to a powder X-ray diffraction pattern, DSC thermogram, or TGA pattern that is non-identical to those depicted herein, and shares a majority of major peaks, which fall within the limits experimental error, when considered by one of ordinary skill in the art.

### Compound 1

Disclosed herein is 5-(4-((7-cyclopropyl-6-oxo-5,6-dihydro-1,5-naphthyridin-3-yl)methylipiperazin-1-yl)-N-methylpicolinamide (compound 1), or a pharmaceutically acceptable salt of solvate thereof. Compound 1 refers to the compound with the following formula:

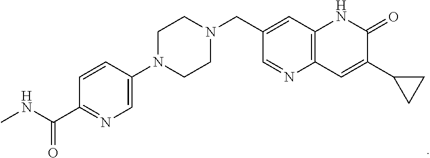 [SMILES: CNC(=O)c1ccc(N2CCN(Cc3cnc4cc(C5CC5)c(=O)[nH]c4c3)CC2)cn1]

Disclosed herein is 5-(4-((7-cyclopropyl-6-oxo-5,6-dihydro-1,5-naphthyridin-3-yl)methyl)piperazin-1-yl)-N-methylpicolinamide (compound 1), or a pharmaceutically acceptable solvate thereof. Disclosed herein is 5-(4-((7-cyclopropyl-6-oxo-5,6-dihydro-1,5-naphthyridin-3-yl)methylipiperazin-1-yl)-N-methylpicolinamide (compound 1).

In some embodiments, compound 1 is a freebase.

In some embodiments, compound 1 is a solvate. In some embodiments, compound 1 is a hydrate. In some embodiments, compound 1 is unsolvated. In some embodiments, compound 1 is anhydrous.

While not intending to be bound by any particular theory, certain solid forms are characterized by physical properties, e.g., stability, solubility, and dissolution rate, appropriate for pharmaceutical and therapeutic dosage forms. Moreover, while not wishing to be bound by any particular theory, certain solid forms are characterized by physical properties (e.g., density, compressibility, hardness, morphology, cleavage, stickiness, solubility, water uptake, electrical properties, thermal behavior, solid-state reactivity, physical stability, and chemical stability) affecting particular processes (e.g., yield, filtration, washing, drying, milling, mixing, tableting, flowability, dissolution, formulation, and lyophilization) which make certain solid forms suitable for the manufacture of a solid dosage form. Such properties can be determined using particular analytical chemical techniques, including solid-state analytical techniques (e.g., X-ray diffraction, microscopy, spectroscopy, and thermal analysis), as described herein.

### Crystalline Forms

The identification and selection of a solid form of a pharmaceutical compound are complex, given that a change in solid form may affect a variety of physical and chemical properties, which may provide benefits or drawbacks in processing, formulation, stability, bioavailability, storage, and handling (e.g., shipping), among other important pharmaceutical characteristics. Useful pharmaceutical solids include crystalline solids and amorphous solids, depending on the product and its mode of administration. Amorphous solids are characterized by a lack of long-range structural order, whereas crystalline solids are characterized by structural periodicity. The desired class of pharmaceutical solid depends upon the specific application; amorphous solids are sometimes selected on the basis of, e.g., an enhanced dissolution profile, while crystalline solids may be desirable for properties such as, e.g., physical, or chemical stability.

Whether crystalline or amorphous, solid forms of a pharmaceutical compound include single-component and multiple-component solids. Single-component solids consist essentially of the pharmaceutical compound or active ingredient in the absence of other compounds. Variety among single-component crystalline materials may potentially arise from the phenomenon of polymorphism, wherein multiple three-dimensional arrangements exist for a particular pharmaceutical compound.

Notably, it is not possible to predict a priori if crystalline forms of a compound even exist, let alone how to successfully prepare them (see, e.g., Braga and Grepioni, 2005, “Making crystals from crystals: a green route to crystal engineering and polymorphism,” Chem. Commun.:3635-3645 (with respect to crystal engineering, if instructions are not very precise and/or if other external factors affect the process, the result can be unpredictable); Jones et al., 2006, Pharmaceutical Cocrystals: An Emerging Approach to Physical Property Enhancement,” MRS Bulletin 31:875-879 (At present it is not generally possible to computationally predict the number of observable polymorphs of even the simplest molecules); Price, 2004, “The computational prediction of pharmaceutical crystal structures and polymorphism,” Advanced Drug Delivery Reviews 56:301-319 (“Price”); and Bernstein, 2004, “Crystal Structure Prediction and Polymorphism,” ACA Transactions 39:14-23 (a great deal still needs to be learned and done before one can state with any degree of confidence the ability to predict a crystal structure, much less polymorphic forms)).

The variety of possible solid forms creates potential diversity in physical and chemical properties for a given pharmaceutical compound. The discovery and selection of solid forms are of great importance in the development of an effective, stable, and marketable pharmaceutical product.

### Crystalline Forms of Compound 1

The polymorphs made according to the methods of the invention may be characterized by any methodology according to the art. For example, the polymorphs made according to the methods of the invention may be characterized by X-ray powder diffraction (XRPD), differential scanning calorimetry (DSC), thermogravimetric analysis (TGA), hot-stage microscopy, and/or spectroscopy (e.g., Raman, solid state nuclear magnetic resonance (ssNMR), and infrared (IR)). In some embodiments, crystallinity of a solid form is determined by X-Ray Powder Diffraction (XRPD).

XRPD: Polymorphs according to the invention may be characterized by XRPD. The relative intensities of XRPD peaks can vary, depending upon the particle size, the sample preparation technique, the sample mounting procedure and the particular instrument employed. Moreover, instrument variation and other factors can affect the 20 values. Therefore, the XRPD peak assignments can vary, for example by plus or minus 0.2 degrees.

DSC: Polymorphs according to the invention can also be identified by its characteristic DSC thermograms. For DSC, it is known that the temperatures observed will depend upon the rate of temperature change as well as sample preparation technique and the particular instrument employed. Thus, the values reported herein relating to DSC thermograms can vary, for example by plus or minus 4° C.

TGA: The polymorphic forms of the invention may also give rise to thermal behavior different from that of the amorphous material or another polymorphic form. Thermal behavior may be measured in the laboratory by thermogravimetric analysis (TGA) which may be used to distinguish some polymorphic forms from others. In one aspect, the polymorph may be characterized by thermogravimetric analysis.

The polymorph forms of compound 1 are useful in the production of medicinal preparations and can be obtained by means of a crystallization process to produce crystalline and semi-crystalline forms or a solidification process to obtain the amorphous form. In some embodiments, the crystallization is carried out by either generating the desired compound (for example, compound 1) in a reaction mixture and isolating the desired polymorph from the reaction mixture, or by dissolving raw compound in a solvent, optionally with heat, followed by crystallizing/solidifying the product by cooling (including active cooling) and/or by the addition of an antisolvent for a period of time. In some embodiments, the crystallization comprises addition of a seed form of a desired polymorph. The crystallization or solidification may be followed by drying carried out under controlled conditions until the desired water content is reached in the end polymorphic form.

### Polymorph Form I of Compound 1

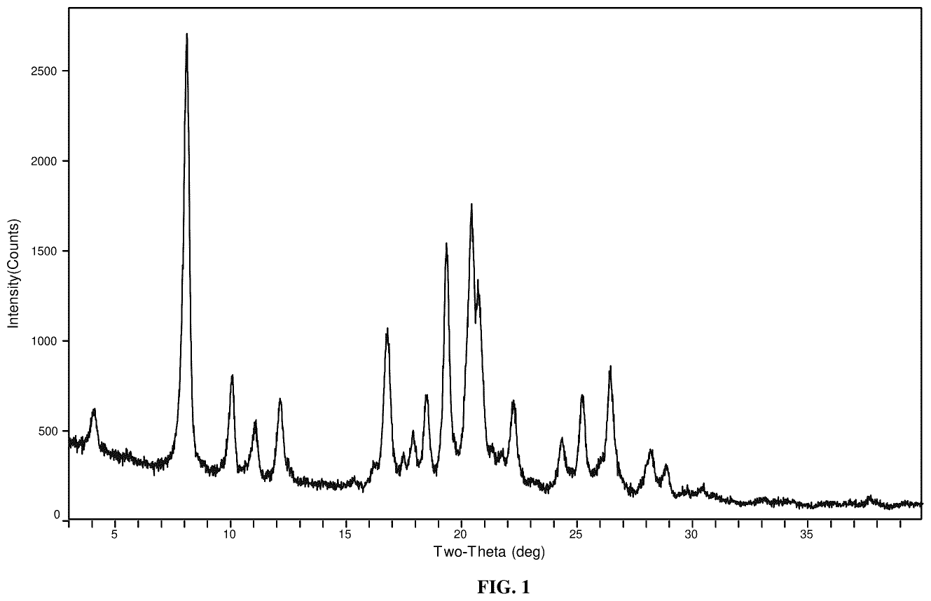

In some embodiments, compound 1 is crystalline. In some embodiments, crystalline compound 1 is Form I characterized as having at least one of the following properties:
(a) an X-ray powder diffraction (XRPD) pattern substantially the same as shown in FIG. 1;
(b) an X-ray powder diffraction (XRPD) pattern with characteristic peaks at 8.11±0.2° 2θ, 16.80±0.2° 2θ, 19.35±0.2° 2θ, 20.44±0.2° 2θ, and 24.46±0.2° 2θ.
(c) a DSC thermogram with an endotherm having a peak temperature at about 255° C.;
(d) a DSC thermogram with an endotherm having a peak temperature at about 258° C.;
(e) a DSC thermogram with an endotherm having a peak temperature at about 266° C.;
(f) a thermogravimetric analysis (TGA) thermogram comprising a loss in mass of about 1.4% over a temperature range of about 70° C. to about 255° C.; or p1 (g) combinations thereof.

In some embodiments, crystalline compound 1, Form I is characterized as having at least one of the properties selected from (a) to (f). In some embodiments, crystalline compound 1, Form I is characterized as having at least two of the properties selected from (a) to (f). In some embodiments, crystalline compound 1, Form I is characterized as having at least three of the properties selected from (a) to (f). In some embodiments, crystalline compound 1, Form I is characterized as having at least four of the properties selected from (a) to (f). In some embodiments, crystalline compound 1, Form I is characterized as having at least five of the properties selected from (a) to (f). In some embodiments, crystalline compound 1, Form I is characterized as having properties (a) to (f).

In some embodiments, crystalline compound 1, Form I has an X-ray powder diffraction (XRPD) pattern substantially the same as shown in FIG. 1.

In some embodiments, crystalline compound 1, Form I has an X-ray powder diffraction (XRPD) pattern with characteristic peaks found in Table 1.

In some embodiments, crystalline compound 1, Form I has an X-ray powder diffraction (XRPD) pattern with characteristic peaks at 8.11±0.2° 2θ, 16.80±0.2° 2θ, 19.35±0.2° 2θ, 20.44±0.2° 2θ, and 24.46±0.2° 2θ.

In some embodiments, crystalline compound 1, Form I has an X-ray powder diffraction (XRPD) pattern with at least three characteristic peaks selected from 8.11±0.2° 2θ, 16.80±0.2° 2θ, 19.35±0.2° 2θ, 20.44±0.2° 2θ, and 24.46±0.2° 2θ.

In some embodiments, crystalline compound 1, Form I has an X-ray powder diffraction (XRPD) pattern with at least four characteristic peaks selected from 8.11±0.2° 2θ, 16.80±0.2° 2θ, 19.35±0.2° 2θ, 20.44±0.2° 2θ, and 24.46±0.2° 2θ.

In some embodiments, the X-ray powder diffraction pattern further comprises at least one peak selected from 10.10±0.2° 2θ, 12.14±0.2° 2θ, 22.25±0.2° 2θ, and 25.22±0.2° 2θ.

In some embodiments, the X-ray powder diffraction pattern further comprises at least two peaks selected from 10.10±0.2° 2θ, 12.14±0.2° 2θ, 22.25±0.2° 2θ, and 25.22±0.2° 2θ.

In some embodiments, the X-ray powder diffraction pattern further comprises at least three peaks selected from 10.10±0.2° 2θ, 12.14±0.2° 2θ, 22.25±0.2° 2θ, and 25.22±0.2° 2θ.

In some embodiments, the X-ray powder diffraction pattern further comprises peaks at 10.10±0.2° 2θ, 12.14±0.2° 2θ, 22.25±0.2° 2θ, and 25.22±0.2° 2θ.

In some embodiments, crystalline compound 1, Form I has an X-ray powder diffraction (XRPD) pattern with characteristic peaks at 8.11±0.2° 2θ, 10.10±0.2° 2θ, 12.14±0.2° 2θ, 16.80±0.2° 2θ, 19.35±0.2° 2θ, 20.44±0.2° 2θ, 22.25±0.2° 2θ, 24.46±0.2° 2θ, and 25.22±0.2° 2θ.

In some embodiments, crystalline compound 1, Form I has an X-ray powder diffraction (XRPD) pattern with at least five characteristic peaks selected from 8.11±0.2° 2θ, 10.10±0.2° 2θ, 12.14±0.2° 2θ, 16.80±0.2° 2θ, 19.35±0.2° 2θ, 20.44±0.2° 2θ, 22.25±0.2° 2θ, 24.46±0.2° 2θ, and 25.22±0.2° 2θ.

In some embodiments, crystalline compound 1, Form I has an X-ray powder diffraction (XRPD) pattern with at least six characteristic peaks selected from 8.11±0.2° 2θ, 10.10±0.2° 2θ, 12.14±0.2° 2θ, 16.80±0.2° 2θ, 19.35±0.2° 2θ, 20.44±0.2° 2θ, 22.25±0.2° 2θ, 24.46±0.2° 2θ, and 25.22±0.2° 2θ.

In some embodiments, crystalline compound 1, Form I has an X-ray powder diffraction (XRPD) pattern with at least seven characteristic peaks selected from 8.11±0.2° 2θ, 10.10±0.2° 2θ, 12.14±0.2° 2θ, 16.80±0.2° 2θ, 19.35±0.2° 2θ, 20.44±0.2° 2θ, 22.25±0.2° 2θ, 24.46±0.2° 2θ, and 25.22±0.2° 2θ.

In some embodiments, crystalline compound 1, Form I has an X-ray powder diffraction (XRPD) pattern with at least eight characteristic peaks selected from 8.11±0.2° 2θ, 10.10±0.2° 2θ, 12.14±0.2° 2θ, 16.80±0.2° 2θ, 19.35±0.2° 2θ, 20.44±0.2° 2θ, 22.25±0.2° 2θ, 24.46±0.2° 2θ, and 25.22±0.2° 2θ.

In some embodiments, the X-ray powder diffraction pattern further comprises at least one peak selected from 17.90±0.2° 2θ, 24.36±0.2° 2θ, and 28.18±0.2° 2θ.

In some embodiments, crystalline compound 1, Form I has a DSC thermogram with an endotherm having a peak temperature at about 255° C.

In some embodiments, crystalline compound 1, Form I has a DSC thermogram with an endotherm having a peak temperature at about 258° C.

In some embodiments, crystalline compound 1, Form I has a DSC thermogram with an endotherm having a peak temperature at about 266° C.

In some embodiments, crystalline compound 1, Form I has a thermogravimetric analysis (TGA) thermogram comprising a loss in mass of about 1.4% over a temperature range of about 70° C. to about 255° C.

In some embodiments, crystalline compound 1, Form I is anhydrous.

TABLE 1
Form I
Form I

| 2-Theta | d(Å) | BG | Height | I % | Area | I % | FWHM |
|---|---|---|---|---|---|---|---|
| 4.116 | 21.4491 | 400 | 220 | 9.1 | 5269 | 7.8 | 0.267 |
| 8.107 | 10.8964 | 286 | 2428 | 100 | 67530 | 100 | 0.31 |
| 10.09 | 8.7595 | 281 | 526 | 21.7 | 11348 | 16.8 | 0.241 |
| 11.1 | 7.9645 | 265 | 292 | 12 | 5934 | 8.8 | 0.227 |
| 12.139 | 7.2852 | 225 | 451 | 18.6 | 12566 | 18.6 | 0.311 |
| 15.366 | 5.7617 | 177 | 66 | 2.7 | 1540 | 2.3 | 0.26 |
| 16.171 | 5.4764 | 213 | 115 | 4.7 | 3178 | 4.7 | 0.308 |
| 16.8 | 5.273 | 242 | 827 | 34.1 | 24582 | 36.4 | 0.332 |
| 17.495 | 5.0649 | 284 | 92 | 3.8 | 812 | 1.2 | 0.099 |
| 17.903 | 4.9504 | 278 | 219 | 9 | 6031 | 8.9 | 0.307 |
| 18.455 | 4.8036 | 290 | 407 | 16.8 | 8485 | 12.6 | 0.233 |
| 19.347 | 4.584 | 325 | 1219 | 50.2 | 27188 | 40.3 | 0.249 |
| 20.437 | 4.342 | 365 | 1399 | 57.6 | 60357 | 89.4 | 0.482 |
| 20.752 | 4.2768 | 355 | 905 | 37.3 | 36809 | 54.5 | 0.454 |
| 21.721 | 4.0882 | 318 | 66 | 2.7 | 1512 | 2.2 | 0.256 |
| 22.249 | 3.9922 | 286 | 380 | 15.7 | 8117 | 12 | 0.238 |
| 24.362 | 3.6506 | 224 | 234 | 9.6 | 5408 | 8 | 0.258 |
| 25.216 | 3.5288 | 263 | 434 | 17.9 | 9280 | 13.7 | 0.239 |
| 26.018 | 3.4219 | 254 | 96 | 4 | 4883 | 7.2 | 0.568 |
| 26.463 | 3.3653 | 217 | 641 | 26.4 | 18399 | 27.2 | 0.32 |
| 28.184 | 3.1636 | 152 | 240 | 9.9 | 9062 | 13.4 | 0.421 |
| 28.853 | 3.0918 | 186 | 124 | 5.1 | 2183 | 3.2 | 0.196 |
| 30.493 | 2.9291 | 126 | 76 | 3.1 | 1786 | 2.6 | 0.262 |
| 37.731 | 2.3822 | 79 | 56 | 2.3 | 1143 | 1.7 | 0.228 |

### Polymorph Form II of Compound 1

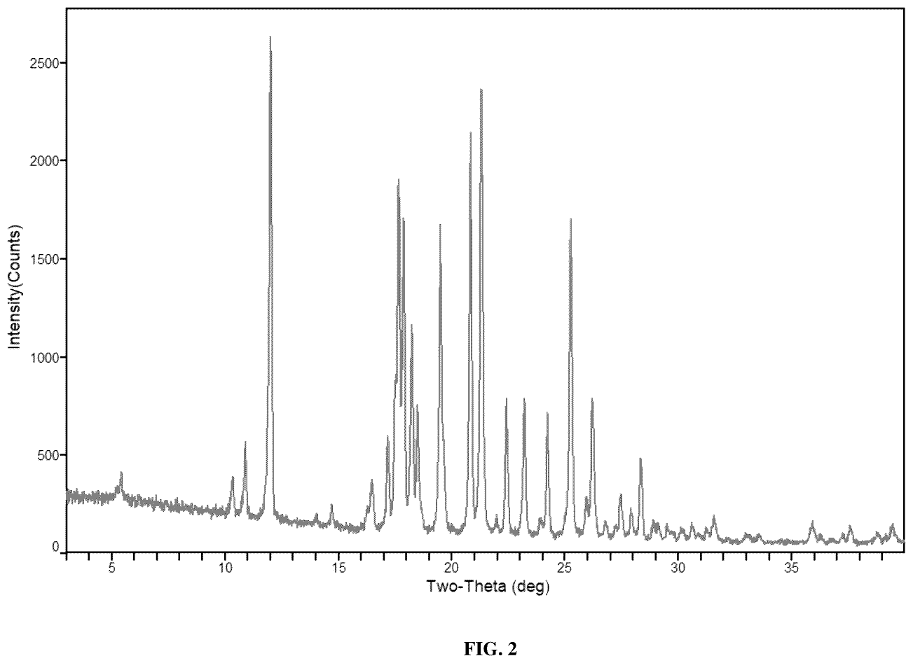

In some embodiments, compound 1 is crystalline. In some embodiments, crystalline compound 1 is Form II characterized as having at least one of the following properties:
(a) an X-ray powder diffraction (XRPD) pattern substantially the same as shown in FIG. 2;
(b) an X-ray powder diffraction (XRPD) pattern with characteristic peaks at 20.88±0.2° 2θ, 21.37±0.2° 2θ, and ±25.31 0.2° 2θ.
(c) a DSC thermogram with an endotherm having an onset temperature at about 265.5° C. and a peak temperature at about 266.5° C.; or
(d) combinations thereof.

In some embodiments, crystalline compound 1, Form II is characterized as having at least one of the properties selected from (a) to (c). In some embodiments, crystalline compound 1, Form II is characterized as having at least two of the properties selected from (a) to (c). In some embodiments, crystalline compound 1, Form II is characterized as having properties (a) to (c).

In some embodiments, crystalline compound 1, Form II has an X-ray powder diffraction (XRPD) pattern substantially the same as shown in FIG. 2.

In some embodiments, crystalline compound 1, Form II has an X-ray powder diffraction (XRPD) pattern with characteristic peaks found in Table 2.

In some embodiments, crystalline compound 1, Form II has an X-ray powder diffraction (XRPD) pattern with characteristic peaks at 20.88±0.2° 2θ, 21.37±0.2° 2θ, and ±25.31 0.2° 2θ.

In some embodiments, the X-ray powder diffraction pattern further comprises a peak at 10.96±0.2° 2θ.

In some embodiments, the X-ray powder diffraction pattern further comprises a peak at 16.57±0.2° 2θ.

In some embodiments, the X-ray powder diffraction pattern further comprises a peak at 17.26±0.2° 2θ.

In some embodiments, the X-ray powder diffraction pattern further comprises a peak at 17.71±0.2° 2θ.

In some embodiments, the X-ray powder diffraction pattern further comprises a peak at 17.93±0.2° 2θ.

In some embodiments, the X-ray powder diffraction pattern further comprises a peak at 19.54±0.2° 2θ.

In some embodiments, the X-ray powder diffraction pattern further comprises a peak at 23.25±0.2° 2θ.

In some embodiments, the X-ray powder diffraction pattern further comprises a peak at 26.27±0.2° 2θ.

In some embodiments, crystalline compound 1, Form II has an X-ray powder diffraction (XRPD) pattern with characteristic peaks at 10.96±0.2° 2θ, 16.57±0.2° 2θ, 17.26±0.2° 2θ, 17.71±0.2° 2θ, 17.93±0.2° 2θ, 19.54±0.2° 2θ, 20.88±0.2° 2θ, 21.37±0.2° 2θ, 23.25±0.2° 2θ, 25.31 0.2° 2θ, and 26.27±0.2° 2θ.

In some embodiments, crystalline compound 1, Form II has an X-ray powder diffraction (XRPD) pattern with at least five characteristic peaks selected from 10.96±0.2° 2θ, 16.57±0.2° 2θ, 17.26±0.2° 2θ, 17.71±0.2° 2θ, 17.93±0.2° 2θ, 19.54±0.2° 2θ, 20.88±0.2° 2θ, 21.37±0.2° 2θ, 23.25±0.2° 2θ, 25.31 0.2° 2θ, and 26.27±0.2° 2θ.

In some embodiments, crystalline compound 1, Form II has an X-ray powder diffraction (XRPD) pattern with at least six characteristic peaks selected from 10.96±0.2° 2θ, 16.57±0.2° 2θ, 17.26±0.2° 2θ, 17.71±0.2° 2θ, 17.93±0.2° 2θ, 19.54±0.2° 2θ, 20.88±0.2° 2θ, 21.37±0.2° 2θ, 23.25±0.2° 2θ, 25.31 0.2° 2θ, and 26.27±0.2° 2θ.

In some embodiments, crystalline compound 1, Form II has an X-ray powder diffraction (XRPD) pattern with at least seven characteristic peaks selected from 10.96±0.2° 2θ, 16.57±0.2° 2θ, 17.26±0.2° 2θ, 17.71±0.2° 2θ, 17.93±0.2° 2θ, 19.54±0.2° 2θ, 20.88±0.2° 2θ, 21.37±0.2° 2θ, 23.25±0.2° 2θ, 25.31 0.2° 2θ, and 26.27±0.2° 2θ.

In some embodiments, crystalline compound 1, Form II has an X-ray powder diffraction (XRPD) pattern with at least eight characteristic peaks selected from 10.96±0.2° 2θ, 16.57±0.2° 2θ, 17.26±0.2° 2θ, 17.71±0.2° 2θ, 17.93±0.2° 2θ, 19.54±0.2° 2θ, 20.88±0.2° 2θ, 21.37±0.2° 2θ, 23.25±0.2° 2θ, 25.31 0.2° 2θ, and 26.27±0.2° 2θ.

In some embodiments, crystalline compound 1, Form II has a DSC thermogram with an endotherm having an onset temperature at about 265.5° C. and a peak temperature at about 266.5° C.

In some embodiments, crystalline compound 1, Form II is anhydrous.

In some embodiments, crystalline compound 1, Form II is physically and chemically stable.

In some embodiments, crystalline compound 1, Form II is physically and chemically stable at both 60° C. (capped) and 40° C./75% RH (open) after 7 days.

TABLE 2
Form II
Form II

| 2-Theta | d(Å) | BG | Height | I % | Area | I % | FWHM |
|---|---|---|---|---|---|---|---|
| 5.49 | 16.0838 | 264 | 156 | 8 | 2546 | 9.1 | 0.182 |
| 10.367 | 8.5256 | 182 | 183 | 9.4 | 2376 | 8.5 | 0.145 |
| 10.957 | 8.0679 | 197 | 309 | 15.9 | 2915 | 10.4 | 0.105 |
| 12.072 | 7.3251 | 163 | 1880 | 96.5 | 24424 | 87.5 | 0.145 |
| 14.068 | 6.29 | 134 | 51 | 2.6 | 863 | 3.1 | 0.189 |
| 14.802 | 5.9797 | 125 | 151 | 7.7 | 1849 | 6.6 | 0.137 |
| 16.355 | 5.4155 | 120 | 117 | 6 | 2093 | 7.5 | 0.2 |
| 16.575 | 5.3439 | 121 | 244 | 12.5 | 4275 | 15.3 | 0.196 |
| 17.257 | 5.1342 | 206 | 399 | 20.5 | 3736 | 13.4 | 0.105 |
| 17.718 | 5.0016 | 156 | 1206 | 61.9 | 27922 | 100 | 0.258 |
| 17.928 | 4.9435 | 122 | 1061 | 54.4 | 18814 | 67.4 | 0.198 |
| 18.297 | 4.8448 | 111 | 888 | 45.6 | 14254 | 51 | 0.179 |
| 18.559 | 4.7768 | 111 | 413 | 21.2 | 7382 | 26.4 | 0.199 |
| 19.546 | 4.5379 | 111 | 1052 | 54 | 16700 | 59.8 | 0.177 |
| 20.883 | 4.2503 | 108 | 1436 | 73.7 | 18087 | 64.8 | 0.141 |
| 21.369 | 4.1546 | 103 | 1949 | 100 | 27713 | 99.3 | 0.159 |
| 22.026 | 4.0323 | 102 | 83 | 4.3 | 923 | 3.3 | 0.124 |
| 22.46 | 3.9552 | 98 | 522 | 26.8 | 6216 | 22.3 | 0.133 |
| 23.248 | 3.823 | 90 | 576 | 29.6 | 7996 | 28.6 | 0.155 |
| 23.981 | 3.7077 | 87 | 72 | 3.7 | 1083 | 3.9 | 0.168 |
| 24.285 | 3.6621 | 84 | 459 | 23.6 | 6499 | 23.3 | 0.158 |
| 25.31 | 3.516 | 86 | 1254 | 64.3 | 18458 | 66.1 | 0.164 |
| 26.029 | 3.4205 | 84 | 162 | 8.3 | 3129 | 11.2 | 0.216 |
| 26.267 | 3.39 | 80 | 540 | 27.7 | 9145 | 32.8 | 0.189 |
| 26.834 | 3.3196 | 78 | 57 | 2.9 | 423 | 1.5 | 0.083 |
| 27.527 | 3.2376 | 77 | 173 | 8.9 | 2356 | 8.4 | 0.152 |
| 27.988 | 3.1854 | 67 | 123 | 6.3 | 2102 | 7.5 | 0.191 |
| 28.42 | 3.1378 | 68 | 304 | 15.6 | 4428 | 15.9 | 0.163 |
| 28.971 | 3.0794 | 63 | 47 | 2.4 | 1059 | 3.8 | 0.251 |
| 29.181 | 3.0577 | 63 | 86 | 4.4 | 1506 | 5.4 | 0.195 |
| 29.626 | 3.0129 | 60 | 84 | 4.3 | 1554 | 5.6 | 0.206 |
| 30.259 | 2.9513 | 62 | 52 | 2.7 | 615 | 2.2 | 0.132 |
| 30.689 | 2.9108 | 70 | 84 | 4.3 | 553 | 2 | 0.073 |
| 31.28 | 2.8572 | 55 | 45 | 2.3 | 1854 | 6.6 | 0.46 |
| 31.702 | 2.8201 | 56 | 90 | 4.6 | 2241 | 8 | 0.278 |
| 33.069 | 2.7066 | 45 | 51 | 2.6 | 935 | 3.3 | 0.205 |
| 33.632 | 2.6626 | 42 | 54 | 2.8 | 1325 | 4.7 | 0.274 |
| 35.97 | 2.4947 | 41 | 73 | 3.7 | 1567 | 5.6 | 0.24 |
| 36.321 | 2.4714 | 40 | 39 | 2 | 661 | 2.4 | 0.189 |
| 37.307 | 2.4083 | 40 | 37 | 1.9 | 1020 | 3.7 | 0.308 |
| 37.665 | 2.3862 | 38 | 62 | 3.2 | 1748 | 6.3 | 0.315 |
| 38.873 | 2.3148 | 43 | 53 | 2.7 | 1482 | 5.3 | 0.312 |
| 39.567 | 2.2758 | 46 | 73 | 3.7 | 1795 | 6.4 | 0.274 |

### Preparation of Crystalline Compound 1

In some embodiments, crystalline forms of compound 1 are prepared as outlined in the Examples. It is noted that solvents, temperatures, and other reaction conditions presented herein may vary.

In some embodiments, provided herein are methods for making a solid form of compound 1, comprising 1) suspending compound 1 in a solvent at a first temperature (e.g., ambient temperature); 2) cycling the compound 1 mixture between ambient and a second temperature (e.g., about 40° C.); 3) collecting a solid if there is precipitation, or evaporating the solvent to collect a solid if there is no precipitation; and 4) optionally drying. In some embodiments, provided herein are methods for making a solid form of compound 1, comprising 1) obtaining a saturated solution of compound 1 in a solvent; 2) adding an anti-solvent into the saturated solution; 3) cooling down to about 2-8° C. and about -20° C.; 4) collecting a solid if there is precipitation, or evaporating the solvent to collect a solid if there is no precipitation; and 5) optionally drying. In some embodiments, the ratio by volume of solvent and anti-solvent is about 1:9. In some embodiments, the ratio by volume of solvent and anti-solvent is about 1:4. In some embodiments, the ratio by volume of solvent and anti-solvent is about 1:2. In some embodiments, the ratio by volume of solvent and anti-solvent is about 1:1. In some embodiments, the methods for making a solid form of compound 1 are anti-solvent recrystallization experiments.

In another embodiment, crystalline compound 1 is substantially pure. In some embodiments, the substantially pure crystalline compound 1. In some embodiments, the pure crystalline compound 1 is substantially free of other solid forms, e.g., amorphous solid. In some embodiments, the purity of the substantially pure crystalline compound 1 is no less than about 95%, no less than about 96%, no less than about 97%, no less than about 98%, no less than about 98.5%, no less than about 99%, no less than about 99.5%, or no less than about 99.8%. In some embodiments, the purity of the substantially pure crystalline compound 1 is about 95%, about 96%, about 97%, about 98%, about 98.5%, about 99%, about 99.5%, or about 99.8%.

### Method of Treatment

Disclosed herein are methods of treatment of a disease in which inhibition of PARP is beneficial, the method comprising administering a compound disclosed herein. Also disclosed herein are methods of treatment of a disease in which inhibition of PARP1 is beneficial, the method comprising administering a compound disclosed herein. In some embodiments, the disease is cancer. In some embodiments, the cancer is breast cancer, ovarian cancer, pancreatic cancer, prostate cancer, a hematological cancer, a gastrointestinal cancer such as gastric cancer and colorectal cancer, or lung cancer. In some embodiments, the cancer is breast cancer, ovarian cancer, pancreatic cancer, or prostate cancer. In some embodiment, the cancer is leukemia, colon cancer, glioblastoma, lymphoma, melanoma, or cervical cancer.

In some embodiments, the cancer comprises a BRCA1 and/or a BRCA2 mutation.

In some embodiments, the cancer comprising a BRCA1 and/or a BRCA2 mutation is bladder cancer, brain & CNS cancers, breast cancer, cervical cancer, colorectal cancer, esophagus cancer, Hodgkin lymphoma, non-Hodgkin lymphoma, kidney cancer, leukemia, lung cancer, melanoma, myeloma, oral cavity cancer, ovarian cancer, pancreatic cancer, prostate cancer, skin cancer, stomach cancer, thyroid cancer, or uterus cancer.

In some embodiments, the cancer is a cancer deficient in Flomologous Recombination (FIR) dependent DNA DSB repair activity. The FIR dependent DNA DSB repair pathway repairs double-strand breaks (DSBs) in DNA via homologous mechanisms to reform a continuous DNA helix. The components of the FIR dependent DNA DSB repair pathway include, but are not limited to, ATM (NM_000051), RAD51 (NM_002875), RAD51 L1 (NM_002877), RAD51 C (NM_002876), RAD51 L3 (NM_002878), DMC1 (NM_007068), XRCC2 (NM_005431), XRCC3 (NM_005432), RAD52 (NM_002879), RAD54L (NM_003579), RAD54B (NM_012415), BRCA1 (NM_007295), BRCA2 (NM_000059), RAD50 (NM_005732), MRE1 1 A (NM_005590) and NBS1 (NM_002485). Other proteins involved in the FIR dependent DNA DSB repair pathway include regulatory factors such as EMSY. In some embodiments, the cancer which is deficient in FIR dependent DNA DSB repair comprises one or more cancer cells which have a reduced or abrogated ability to repair DNA DSBs through that pathway, relative to normal cells i.e. the activity of the FIR dependent DNA DSB repair pathway may be reduced or abolished in the one or more cancer cells.

In some embodiments, the activity of one or more components of the FIR dependent DNA DSB repair pathway is abolished in the one or more cancer cells of an individual having a cancer which is deficient in FIR dependent DNA DSB repair.

In some embodiments, the cancer cells have a BRCA1 and/or a BRCA2 deficient phenotype i.e. BRCA1 and/or BRCA2 activity is reduced or abolished in the cancer cells. Cancer cells with this phenotype may be deficient in BRCA1 and/or BRCA2, i.e. expression and/or activity of BRCA1 and/or BRCA2 may be reduced or abolished in the cancer cells, for example by means of mutation or polymorphism in the encoding nucleic acid, or by means of amplification, mutation or polymorphism in a gene encoding a regulatory factor, for example the EMSY gene which encodes a BRCA2 regulatory factor. BRCA1 and BRCA2 are known tumor suppressors whose wild-type alleles are frequently lost in tumors of heterozygous carriers. Amplification of the EMSY gene, which encodes a BRCA2 binding factor, is also known to be associated with breast and ovarian cancer. Carriers of mutations in BRCA1 and/or BRCA2 are also at elevated risk of certain cancers, including breast cancer, ovarian cancer, pancreatic cancer, prostate cancer, a hematological cancer, gastrointestinal cancer, and lung cancer.

To minimize the risks of off-target effects it is desirable for drug molecules to possess selectivity for a specific target.

Avoiding inhibition of PARP family isoforms beyond FARM may be important in minimizing toxicities that may arise from inhibition of non-PARP1 isoforms. The pharmacology of inhibiting PARP isoforms beyond PARP1 may drive toxicities that reduce the therapeutic index for agents that possess lower selectivity's for PARP1 against PARP isoforms. PARP3, like PARP1, plays a role in DNA damage but has also been found to be a key player in the integrity of the mitotic spindle and in telomerase integrity (Boehler, C., Gauthier, L R., Mortusewicz O. et al, Poly(ADP-ribose) polymerase 3 (PARP3), newcomer in cellular response to DNA damage and mitotic progression, PNAS, Jan. 26, 2011, 108 (7) 2783-2788). PARP5A also known as Tankyrase plays key roles in Wnt signaling and telomere length (Kodak, O., Chen, H., Holohan B. et al. Disruption of Wnt/β-Catenin Signaling and Telomeric Shortening Are Inextricable Consequences of Tankyrase inhibition in Human Cells. Mol Cell. Biol. 2015 July; 35(14), 2425-2435). PARP6 is an essential microtubrile-regulatory gene in mice, germ line mutations in PARPO that abrogate the catalytic activity has negative effects on neuronal function in humans (Vermehren-Schmaedick, A., Huang J. Y., Levinson, M. et al. Characterization of PARP6 Function in Knockout Mice and Patients with Developmental Delay. Cells, 2021 June; 10(6), 1289). PARP7 catalytic inhibition causes hyper stimulatory effects on type one interferon producing an autoimmune phenotype (Gozgit, J. M,, Vashincier, M. M., Abo, R. P. et al. PARP7 negatively regulates the type I interferon response in cancer cells and its inhibition triggers antitumor immunity. Volume 39, Issue 9, 13 Septenther 2021. Pages 1214-1226), While the exact function of PARP8 has not been established, its knockout has been shown to induce mitotic and nuclear morphology defects and a decrease in cellular viability (Vyas, S., Chesarone-Cataldo, M., Todarova, T., et al. A Systematic Analysis of the PARP Protein Family Identifies New Functions Critical for Cell Physiology. Nat. Commun. 2013, 4 (1), 22401. PARP10 has been described as a MYC interacting protein with tumor suppressor activities (Yu, M., Schrcek, S., Cerni, C, et al. PARP-10, a novel Myc-interacting protein with poly(ADP-ribose) polymerase activity, inhibits transformation. Oncogene, 2005 volume 24, pages 1982-4993).

### Dosing

In certain embodiments, the compositions containing compound 1, or a pharmaceutically acceptable salt or solvate thereof, are administered for therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In certain embodiments wherein the patient's condition does not improve, upon the doctor's discretion the administration of the compounds are administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, in specific embodiments, the dosage, or the frequency of administration, or both, is reduced, as a function of the symptoms, to a level at which the improved disease, disorder or condition is retained. In certain embodiments, however, the patient requires intermittent or daily treatment on a long-term basis upon any recurrence of symptoms.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-5000 mg per day. In one aspect, doses employed for adult human treatment are from about 1 mg to about 1000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the compound described herein, or a pharmaceutically acceptable salt thereof, are from about 0.01 to about 50 mg/kg per body weight. In some embodiments, the daily dosage, or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens are determined by standard pharmaceutical procedures in cell cultures or experimental animals, including, but not limited to, the determination of the LD₁₀ and the ED₉₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the compounds described herein lies within a range of circulating concentrations that include the ED₅₀ with minimal toxicity. In certain embodiments, the daily dosage range and/or the unit dosage amount varies within this range depending upon the dosage form employed and the route of administration utilized.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered non-systemically or locally to the mammal.

In any of the aforementioned aspects are further embodiments comprising single administrations of the effective amount of compound 1, or a pharmaceutically acceptable salt or solvate thereof, including further embodiments in which (i) the compound is administered once a day; or (ii) the compound is administered to the mammal multiple times over the span of one day.

In any of the aforementioned aspects are further embodiments comprising multiple administrations of the effective amount of compound 1, or a pharmaceutically acceptable salt or solvate thereof, including further embodiments in which (i) the compound is administered continuously or intermittently: as in a single dose; (ii) the time between multiple administrations is every 6 hours; (iii) the compound is administered to the mammal every 8 hours; (iv) the compound is administered to the subject every 12 hours; (v) the compound is administered to the subject every 24 hours.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, compound 1, or a pharmaceutically acceptable salt or solvate thereof, is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

### Pharmaceutical Compositions/Formulations

In some embodiments, compound 1, or a pharmaceutically acceptable salt or solvate thereof, is administered to a subject in need thereof, either alone or in combination with pharmaceutically acceptable carriers, excipients, or diluents, in a pharmaceutical composition, according to standard pharmaceutical practice. In one embodiment, compound 1, or a pharmaceutically acceptable salt or solvate thereof, may be administered to animals. Compound 1, or a pharmaceutically acceptable salt or solvate thereof, can be administered orally or parenterally, including the intravenous, intramuscular, intraperitoneal, subcutaneous, rectal, and topical routes of administration.

In another aspect, provided herein are pharmaceutical compositions comprising compound 1, or a pharmaceutically acceptable salt or solvate thereof, and at least one pharmaceutically acceptable excipient. Pharmaceutical compositions are formulated in a conventional manner using one or more pharmaceutically acceptable excipients that facilitate processing of the active compounds into preparations that can be used pharmaceutically. Proper formulation is dependent upon the route of administration chosen. A summary of pharmaceutical compositions described herein can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the pharmaceutically acceptable excipient is selected from carriers, binders, filling agents, suspending agents, flavoring agents, sweetening agents, disintegrating agents, dispersing agents, surfactants, lubricants, colorants, diluents, solubilizers, moistening agents, plasticizers, stabilizers, penetration enhancers, wetting agents, anti-foaming agents, antioxidants, preservatives, and any combinations thereof.

The pharmaceutical compositions described herein are administered to a subject by appropriate administration routes, including, but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, liquids, gels, syrups, elixirs, slurries, suspensions, self-emulsifying dispersions, solid solutions, liposomal dispersions, aerosols, solid oral dosage forms, powders, immediate release formulations, controlled release formulations, fast melt formulations, tablets, capsules, pills, powders, dragees, effervescent formulations, lyophilized formulations, delayed release formulations, extended release formulations, pulsatile release formulations, multiparticulate formulations, and mixed immediate and controlled release formulations.

Pharmaceutical compositions including compound 1, or a pharmaceutically acceptable salt or solvate thereof, are manufactured in a conventional manner, such as, by way of example only, by means of conventional mixing, dissolving, granulating, dragee-making, levigating, emulsifying, encapsulating, entrapping, or compression processes.

Pharmaceutical compositions for oral use are obtained by mixing one or more solid excipient with one or more of the compounds described herein, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as polyvinylpyrrolidone (PVP or povidone) or calcium phosphate. If desired, disintegrating agents are added, such as the cross-linked croscarmellose sodium, polyvinylpyrrolidone, agar, or alginic acid or a salt thereof such as sodium alginate. In some embodiments, dyestuffs or pigments are added to the tablets or dragee coatings for identification or to characterize different combinations of active compound doses.

Pharmaceutical compositions that are administered orally include push-fit capsules made of gelatin, as well as soft, sealed capsules made of gelatin and a plasticizer, such as glycerol or sorbitol. The push-fit capsules contain the active ingredients in admixture with filler such as lactose, binders such as starches, and/or lubricants such as talc or magnesium stearate and, optionally, stabilizers. In soft capsules, the active compounds are dissolved or suspended in suitable liquids, such as fatty oils, liquid paraffin, or liquid polyethylene glycols. In some embodiments, stabilizers are added.

Pharmaceutical compositions for parental use are formulated as infusions or injections. In some embodiments, the pharmaceutical composition suitable for injection or infusion includes sterile aqueous solutions, or dispersions, or sterile powders comprising a compound described herein, or a pharmaceutically acceptable salt, solvate, N-oxide, or stereoisomer thereof. In some embodiments, the pharmaceutical composition comprises a liquid carrier. In some embodiments, the liquid carrier is a solvent or liquid dispersion medium comprising, for example, water, saline, ethanol, a polyol (for example, glycerol, propylene glycol, liquid polyethylene glycols, and the like), vegetable oils, nontoxic glyceryl esters, and any combinations thereof. In some embodiments, the pharmaceutical compositions further comprise a preservative to prevent growth of microorganisms.

In some embodiments, the pharmaceutical composition comprises compound 1, wherein in at least 95%, or at least 97%, or at least 99% of compound 1 is Form I of compound 1. In some embodiments, the pharmaceutical composition comprises compound 1, wherein in at least 95% of compound 1 is Form I of compound 1. In some embodiments, the pharmaceutical composition comprises compound 1, wherein in at least 95%, or at least 97%, or at least 99% of compound 1 is Form II of compound 1. In some embodiments, the pharmaceutical composition comprises compound 1, wherein in at least 95% of compound 1 is Form II of compound 1.

In some embodiments, the pharmaceutical composition comprises compound 1, wherein in at least 95%, or at least 97%, or at least 99% of compound 1 is freebase Form I of compound 1. In some embodiments, the pharmaceutical composition comprises compound 1, wherein in at least 95% of compound 1 is freebase Form I of compound 1. In some embodiments, the pharmaceutical composition comprises compound 1, wherein in at least 95%, or at least 97%, or at least 99% of compound 1 is freebase Form II of compound 1. In some embodiments, the pharmaceutical composition comprises compound 1, wherein in at least 95% of compound 1 is freebase Form II of compound 1.

### Combination

Disclosed herein are methods of treating cancer using compound 1, or a pharmaceutically acceptable salt solvate thereof, in combination with an additional therapeutic agent.

In some embodiments, the additional therapeutic agent is an anticancer agent.

In some embodiments, the additional therapeutic agent is administered at the same time as compound 1, or a pharmaceutically acceptable salt or solvate thereof. In some embodiments, the additional therapeutic agent and compound 1, or a pharmaceutically acceptable salt or solvate thereof, are administered sequentially. In some embodiments, the additional therapeutic agent is administered less frequently than compound 1, or a pharmaceutically acceptable salt or solvate thereof. In some embodiments, the additional therapeutic agent is administered more frequently than compound 1, or a pharmaceutically acceptable salt or solvate thereof. In some embodiments, the additional therapeutic agent is administered prior to the administration of compound 1, or a pharmaceutically acceptable salt or solvate thereof. In some embodiments, the additional therapeutic agent is administered after the administration of compound 1, or a pharmaceutically acceptable salt or solvate thereof.

### EXAMPLES

### Reagents

### Acids

The acids listed in Table 3 were used for salt screen.

TABLE 3
List of Acids

| No. | Acid |
|---|---|
| 1 | HCl |
| 2 | H₂SO₄ |
| 3 | H₃PO₄ |
| 4 | Fumaric acid |
| 5 | Citric acid |
| 6 | Maleic acid |
| 7 | L-Tartaric acid |
| 8 | L-Malic acid |
| 9 | Succinic acid |

### Solvents

The solvents used for polymorph screen and salt screen are given in Table 4.

TABLE 4
List of Solvents

| # | Solvent |
|---|---|
| 1 | Methanol (MeOH) |
| 2 | Acetone |
| 3 | Ethyl Acetate (EtOAc) |
| 4 | Methyl tert-butyl ether (MTBE) |
| 5 | Tetrahydrofuran (THF) |
| 6 | Anisole |
| 7 | Toluene |
| 8 | n-Heptane |
| 9 | Dichloromethane (DCM) |
| 10 | Acetonitrile (ACN) |
| 11 | Water |
| 12 | DMSO |

### Example 1

### Synthesis of Starting Material

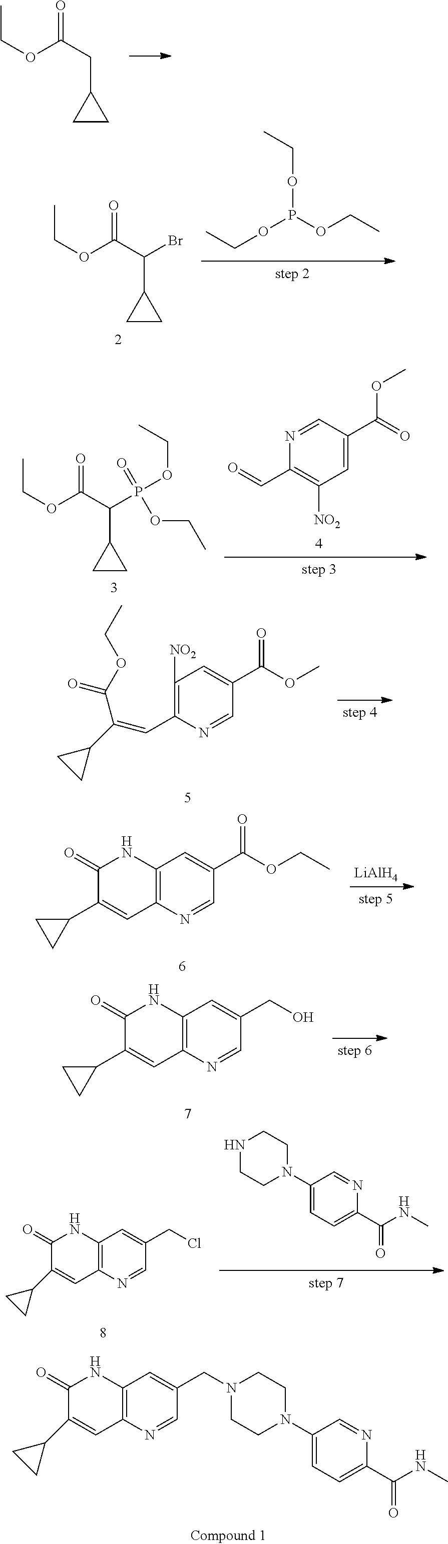

Steps 1-6: Preparation of 7-(chloromethyl)-3-cyclopropyl-1,5-naphthyridin-2(1H)-one

Steps 1-6 are disclosed in PCT/US2022/025357 (WO 2022/225934) example 15.

Step 7: Preparation of 5-(4-((7-cyclopropyl-6-oxo-5,6-dihydro-1,5-naphthyridin-3-ylimethylipiperazin-1-yl)-N-methylpicolinamide (Compound 1):

A mixture of 7-(chloromethyl)-3-cyclopropyl-1,5-naphthyridin-2(1H)-one (16.2 g, 69.0 mmol, 1.00 equiv), N-methyl-5-(piperazin-1-yl)pyridine-2-carboxamide (25.0 g, HCl salt), DIEA (519 g, 345 mmol, 5.00 equiv) and KI (1.14 g, 6.9 mmol, 0.10 equiv) in ACN (100 mL) was stirred for 1 h at 80° C. under nitrogen atmosphere. The reaction was monitored by LCMS. The mixture was allowed to cool down to room temperature. The precipitated solids were collected by filtration and washed with ACN (3×50 mL). The solids were triturated with water (3×50 mL) to afford 5-(4-((7-cyclopropyl-6-oxo-5,6-dihydro-1,5-naphthyridin-3-yl)methyl)piperazin-1-yl)-N-methylpicolinamide (Compound 1, 5.0 g, 97.2% purity) as a brown solid. LC-MS: (ES+H, m/z): [M+H]⁺=419.25. ¹H NMR (300 MHz, DMSO-d₆) δ 11.89 (s, 1H), 8.41-8.35 (m, 2H), 8.26 (d, 1H), 7.82 (d, 1H), 7.61 (d, 1H), 7.44-7.35 (m, 2H), 3.64 (s, 2H), 3.36-3.31 (m, 4H), 2.78 (d, 3H), 2.58-2.53 (m, 4H), 2.22-2.05 (m, 1H), 1.04-0.89 (m, 2H), 0.85-0.80 (m, 2H).

### Physical Characterization

The starting material was received and characterized by XRPD, DSC, TGA, PLM and ¹H-NMR. The characterization data are summarized in Table 5.

The starting material was composed of few irregular crystals and agglomerates, and showed acceptable crystallinity, which was identified as a mixture of Form I and Form II later. About 0.2% ACN and 0.2% DCM was detected by ¹H-NMR. TGA curve showed 1.6% weight loss at 70-290° C., which might be due to loss of solvent. DSC result showed one endothermic peak at 259° C. (onset).

TABLE 5
Characterization Data of Received Batch of Compound 1

| | |
|---|---|
| PLM | Few irregular crystals |
| | and agglomerates |
| XRPD | Freebase Forms I + II |
| DSC Endo Onset/ | 259/264° C., 109 J/g |
| Peak T ΔH | |
| TGA Wt. Loss @T | 1.6% @70-290° C. |
| ¹H-NMR | 0.2% ACN, 0.2% DCM |

### Example 2

### Salt Screening

Compound 1 has a relatively strong base site with calculated pKa of 6.66. Salt screening experiments were conducted with 9 pharmaceutically acceptable acids in 3 solvents. Appropriately 20 mg of the starting material was dispersed in 1.0 mL of MeOH, EtOAc and ACN at RT, respectively. Then 1.1 eq. of selected acid was added, and the mixture was stirred at RT for 1 day. Solid samples were collected by filtration and vacuum drying at 40° C. for 5 hours before XRPD tests. Salt screening results are summarized in Table 6. Seven crystalline salts with acceptable crystallinity were obtained from salt screening experiments.

TABLE 6
Summary of Salt Screening

| | | | Solvent (Vol.) | | |
|---|---|---|---|---|---|
| | | | MeOH | EA | ACN |
| No. | Acid | pKₐ | (50 V) | (50 V) | (50 V) |
| 1 | HCl | −6 | HCl salt | Low | HCl salt |
| | | | Form I | crystallinity | Form II |
| 2 | H₂SO₄ | −3, 1.92 | Sulfate | Freebase | Freebase |
| | | | Form I | Form II | Form II |
| 3 | H₃PO₄ | 1.96 | Freebase | Phosphate | Phosphate |
| | | | Form II + | Form I | Form I |
| | | | peaks | | |
| 4 | Fumaric | 3.03 | Freebase | Fumarate Form | Freebase Form |
| | acid | | Form II | I + freebase | II + fumarate |
| | | | | Form II | |
| 5 | Citric | 3.13 | Freebase | Freebase Form | Freebase Form |
| | acid | | Form II | I + II | I + II |
| 6 | Maleic | 1.92 | Maleate | Maleate | Maleate |
| | acid | | Form I | Form II | Form II |
| 7 | L-Tartaric | 3.02 | Freebase | Low | Low |
| | acid | | Form I + | crystallinity | crystallinity |
| | | | II | | |
| 8 | L-Malic | 3.46 | Freebase | Freebase Form | Freebase |
| | acid | | Form II | I + II | Form I + II |
| 9 | Succinic | 4.21 | Freebase | Low | Low |
| | acid | | Form II | crystallinity | crystallinity |

### HCl Salt

Two crystalline forms of HCl salt with acceptable crystallinity were obtained in MeOH and ACN, assigned as HCl salt Form I and II, respectively. Low crystallinity sample was obtained in EA. HCl salt Form I was obtained in MeOH with high crystallinity. Thermal analysis showed the sample had 4.2% weight loss before 150° C. in TGA. Three broad endothermic peaks at 64° C., 183° C. and 269° C. (onset) and one exothermic peak at 218° C. (onset) were detected in DSC. Negligible residual organic solvent was detected by ¹H-NMR. Hence, HCl salt Form I is postulated to be a hydrate. HCl salt Form II was obtained in ACN with moderate crystallinity. Thermal analysis showed the sample had 2.7% weight loss before 210° C. in TGA. One exothermic peak at 191° C. and one endothermic peak at 252° C. (onset) were detected by DSC. Negligible residual organic solvent was detected by ¹H-NMR. Hence, HCl salt Form II is postulated to be a hydrate.

### Sulfate

Crystalline sulfate was obtained in MeOH with high crystallinity, which was assigned as sulfate Form I. A mixture of freebase Forms I and II was obtained in ACN and EtOAc. Thermal analysis showed the sample had 9.6% weight loss before 150° C. in TGA. Two broad endothermic peaks at 26° C. and 248° C. (onset) were detected in DSC. Negligible residual organic solvent was detected by ¹H-NMR. Hence, sulfate Form I is postulated to be a hydrate.

### Phosphate

Crystalline phosphate was obtained in EtOAc and ACN, and assigned as phosphate Form I. A mixture containing freebase Form II was obtained in MeOH. Phosphate Form I was obtained in ACN with moderate crystallinity and was characterized. The Thermal analysis showed the sample had 4.1% weight loss before 120° C. in TGA. Three broad endothermic peaks at 26° C., 188° C. and 212° C. (onset) were detected in DSC. About 0.3% EtOAc was detected by ¹H-NMR. Hence, phosphate Form I is postulated to be a hydrate.

### Fumarate

Crystalline fumarate was obtained in ACN and EtOAc, but might be mixed with freebase Form II. The sample was obtained in EtOAc with high crystallinity, which was assigned as fumarate Form I. Thermal analysis showed the sample had 1.3% weight loss before 100° C. in TGA curve. One endothermic peaks at 26° C. (onset), two overlapped endothermic peaks at 238° C. (onset) and two exothermic peaks at 167° C. and 220° C. (onset) were detected by DSC. About 0.2% EtOAc was detected by ¹H-NMR and the stoichiometric ration of acid to base was 0.9:1. Therefore, fumarate Form I is postulated to be hydrate

### Citrate

No crystalline citrate but crystalline forms of free base was obtained in salt screening experiments.

### Maleate

Two crystalline forms of maleate with high crystallinity were obtained. which was assigned as maleate Form I and Form II, respectively.

Maleate Form I, obtained in MeOH, was characterized. Thermal analysis showed the sample had three-step weight loss of 2.2% before 100° C., 1.2% at 120-190° C. and 19.1% at 190-250° C. in TGA. Two endothermic peaks at 26° C. and 228° C. (onset), two overlapped endothermic peaks at 263° C. (onset) and one exothermic peak at 140° C. (onset) were detected in DSC. Stoichiometric ratio was determined to be 1:1 by ¹H-NMR, and negligible residual organic solvent was detected. Hence, maleate Form I is postulated to be hydrate.

Maleate Form II, obtained in ACN, was characterized. Thermal analysis showed the sample had 3.1% weight loss before 100° C. and 19.4% weight loss at 150-250° C. in TGA. Four endothermic peaks at 47° C., 135° C., 221° C. and 263° C. (onset) and one exothermic peak at 185° C. (onset) were detected in DSC. Stoichiometric ratio was determined to be 1:1 by ¹H-NMR, and negligible residual organic solvent was detected. Hence, maleate Form II is postulated to be hydrate.

### Tartrate

Low crystallinity samples were obtained in ACN and EtOAc, and freebase Form II was obtained in MeOH.

### Malate

No crystalline malate but crystalline forms of freebase was obtained in salt screening experiments.

### Succinate

Low crystallinity samples were in EtOAc and ACN, and freebase Form II was obtained in MeOH.

### Example 3

### Solubility Estimation

The solubility of compound 1 was estimated at RT by visual observation in 11 solvent systems. Approximately 5 mg solids were weighed into 8 mL glass vial, and then solvent was added stepwise until solids were dissolved completely or a total of solvent volume reached 5 mL. The results are summarized in Table 7. Starting material showed low solubility <5 mg/mL in most tested solvents, except in DMSO. The solubility was estimated and for reference only.

TABLE 7
Estimated Solubility of Starting Material at RT

| | Solubility | |
|---|---|---|
| No. | (mg/mL) | Solvent |
| 1 | 20~50 | DMSO |
| 2 | 1~5 | MeOH, THF (50° C.) |
| 3 | <1 | Acetone, ACN, Toluene, EA, n-Heptane, |
| | | Water, Anisole, DCM, THF |

Values are reported as “<” if dissolution was not observed.

### Example 4

### Polymorph Screening

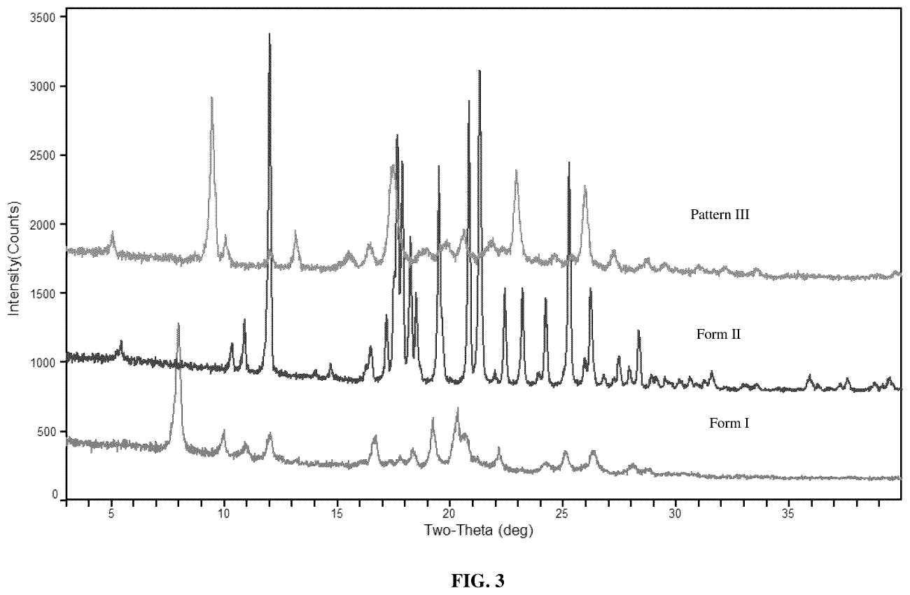

Polymorph screening experiments are summarized in Table 8. A total of three crystalline forms were obtained, including two anhydrates (Forms I and II) and a metastable form (Pattern III). The XRPD patterns are shown in FIG. 3 and the characterization data are summarized in Table 9. The detailed characterization results of each form are presented in the following parts.

TABLE 8
Summary of Polymorph Screening

| No. | Method | Crystalline Form |
|---|---|---|
| 1 | Slurry conversion | Form I, II |
| 2 | Anti-solvent precipitation | Form I, II and Pattern III |
| 3 | Slow evaporation | Form I, II |
| 4 | Quench cooling | Form II |

| | |
|---|---|
| Total | Forms I, II and Pattern III |

TABLE 9
Characterization Data of Compound 1 Polymorphs

| | | DSC Endo | | |
|---|---|---|---|---|
| | | Onset/Peak | TGA | ¹H-NMR |
| Form | Crystallinity | (° C.), | Wt. Loss % | Solvent |
| Solvation | by XRPD | ΔH (J/g) | @T (° C.) | Content |
| Form I | Moderate | 250/255/ | 1.4/70-255 | 0.6% |
| Anhydrate | | 258/266 3 | | MTBE |
| | | adjacent | | |
| | | pks, the | | |
| | | middle is exo | | |
| Form II | High | 266/267, | ~0/RT-200 | 0.1% |
| Anhydrate | | 123 | | MeOH |
| Pattern III | Moderate | N/A | N/A | N/A |
| Metastable | | | | |
| form | | | | |

### Form I

In polymorph screening experiments, pure Form I was obtained via anti-solvent precipitation in DCM/MeOH (1:1, v/v) with MTBE as anti-solvent at RT.

Compound 1 (starting material; 30 mg) was dissolved in 0.8 mL of DCM/MeOH (v/v, 1/1) at room temperature. MTBE (1.2 mL) was added gradually and stirred at room temperature for 5 min. Solid was collected by filtration and dried at 40° C. under vacuum for 5 hours to provide compound 1, Form I.

The XRPD graph is shown in FIG. 1. The XRPD peaks are listed in Table 1. Thermal analysis showed the Form I sample had 1.4% weight loss at 70-255° C. in TGA and three adjacent thermal events in DSC. The two endothermic peaks at 255° C. and 266° C. (peak) were due to melting of Form I and Form II, and the middle exothermic peak at 258° C. was due to recrystallization, which was confirmed by thermal treatment. About 0.6% MTBE was detected by ¹H-NMR. Form I is anhydrous.

In addition, another Form I sample was repeated by the same method. Similarly, three adjacent thermal events were detected by DSC. After heated to 259° C. by DSC, Form I converted to Form II.

### Form H

Form II was obtained in most polymorph screening experiments.

Compound 1 (starting material; 480 g) in H₂O (10 L) was stirred for 5 h at 80° C. The mixture was cooled to room temperature. The solid was collected by filtration and dried at 40° C. to obtain compound 1, Form II.

The XRPD graph is shown in FIG. 2. The XRPD peaks are listed in Table 2. Thermal analysis showed the sample had negligible weight loss before 250° C. in TGA and one sharp endothermic peak at 265° C. (onset) in DSC. About 0.1% MeOH was detected by ¹H-NMR. Hence, Form II is an anhydrate.

### Pattern III

Pattern III was only obtained by anti-solvent precipitation in DMSO with water as anti-solvent. After vacuum drying at 40° C. overnight, Pattern III converted to Form II. Pattern III is a metastable form.

### Example 5

### Scale-Up and Evaluation of Freebase Form II

According to the results of preliminary polymorph screening and salt screening, freebase Form II was scaled up for further evaluation, including hygroscopicity, solubility in bio-relevant media, solid-state stability, and suspension formulation stability.

### Seale-Up and Characterization

Two batches of freebase Form II were successfully scaled up. The product was collected by filtration and dried under vacuum at 40° C. for 3˜5 hours. The detailed information is given in Table 10. Pure Form II was obtained after slurry of starting material in MeOH/acetone and MeOH at RT. Lot#1 was used for solubility and stability study, and Lot#2 was used for additional degradation study in FeSSIF.

The XRPD pattern of sample (Lot#1) was consistent with freebase Form II. This lot sample was fine crystals <5 μm under microscope. Thermal analysis showed the sample had negligible weight loss before 250° C. in TGA and one sharp melting endothermic peak at 264° C. (onset) in DSC. Negligible residual organic solvent was detected by ¹H-NMR. DVS result showed freebase Form II (Lot#1) was non-hygroscopic with water uptake of 0.19% at 80% RH. The crystalline form remained unchanged after DVS test.

The XRPD pattern of sample (Lot#2) was consistent with freebase Form II. This lot sample was fine crystals <5 μm under microscope. Thermal analysis showed the sample had negligible weight loss before 250° C. in TGA and one sharp melting endothermic peak at 264° C. in DSC. Negligible residual organic solvent was detected by ¹H-NMR.

TABLE 10
Preparation of Freebase Form II

| | Amount | Solvent (v:v), | Crystallization | |
|---|---|---|---|---|
| Lot# | (mg) | Vol. | Procedure | Result |
| 1 | 400 | MeOH/acetone | Stirred at RT | Form II |
| | | (3:1), 10 V | for 24 hours | |
| 2 | 300 | MeOH, 10 V | Stirred at RT | Form II |
| | | | overnight | |

### Solubility in Bio-Relevant Media

The solubility of freebase Form II (Lot#1) was measured in bio-relevant media (SGF, FaSSIF and FeSSIF), water and two pH buffers (pH 5.0 and pH 6.5) at 37° C. with 800 rpm for up to 24 hours. About 15 mg of freebase Form II was weighed into sample vials, and then 3 mL of each media was added to make suspensions, respectively. At 0.5, 2 and 24 hours, about 1 mL of each suspension was filtered, the filtrate was analyzed by HPLC and pH, and the filter cake was analyzed by XRPD. Duplicated samples were prepared for each media. The results are summarized in Table 11.

Solubility showed a pH dependence: >5 mg/mL in SGF, ˜50 μg/mL in pH 5.0 buffer, and ˜10 μg/mL in pH 6.5 buffer and water. Compared to control pH buffers, the solubility increased to ˜20 μg/mL in FaSSIF and ˜100 μg/mL in FeSSIF, possibly due to the solubilization effect of bile salt. However, purity of filtrates decreased rapidly in FeSSIF, which suggested a need to investigate the degradation issue with bile salt. Compared to FaSSIF, concentration of bile salt is 5-fold in FeSSIF. The degradation issue in FeSSIF was confirmed with another two batches (Lot #3 and Lot #2) of freebase Form II at 0.5-8 hours, as summarized in Table 12 and Table 13. Several impurities increased rapidly with time. During solubility test, free base Form II remained unchanged in all media.

TABLE 11
Data of Solubility Test in Bio-relevant Media

| Sample (Lot#) | | Initial | Solubility (mg/mL) | | | pH of Filtrate | Filtrate Purity |
|---|---|---|---|---|---|---|---|
| Purity | Medium | pH | 0.5 h | 2 h | 24 h | 0.5/2/24 h | 220 nm-24 h |
| Freebase Form II | FaSSIF | 6.45 | 0.016 | 0.025 | 0.028 | 6.47/6.44/6.40 | — |
| (Lot# 1) | FeSSIF | 4.89 | 0.087 | 0.101 | 0.120 | 4.93/4.90/4.80 | 55% |
| 99.26% | pH 6.5 | 6.43 | 0.007 | 0.008 | 0.010 | 6.42/6.38/6.47 | |
| | buffer | | | | | | |
| | pH 5.0 | 4.94 | 0.045 | 0.050 | 0.061 | 4.85/4.83/4.91 | |
| | buffer | | | | | | |
| | SGF | 1.27 | >5 | >5 | >5 | 1.48 (0.5 h) | 99.28% |
| | Water | 4.18 | 0.007 | 0.008 | 0.010 | 6.98/6.87/6.62 | — |

TABLE 12
Data of Solubility Test in FaSSIF and FeSSIF at 0.5-8 Hours

| Sample (Lot#) | | Initial | Solubility (mg/mL) | pH of Filtrate |
|---|---|---|---|---|
| Purity | Medium | pH | 0.5/2/4/8 h | 0.5/2/4/8 h |
| Freebase Form II | FaSSIF | 6.50 | 0.016/0.017/0.021/0.023 | 6.53/6.45/6.47/6.53 |
| (Lot #3) 99.30% | FeSSIF | 4.98 | 0.085/0.090/0.097/0.105 | 5.00/4.97/5.02/5.04 |
| Freebase Form II | FaSSIF | 6.50 | 0.015/0.015/0.017/0.016 | 6.51/6.47/6.47/6.48 |
| (Lot #2) 99.28% | FeSSIF | 4.98 | 0.087/0.089/0.096/0.101 | 4.99/5.01/5.02/5.01 |

TABLE 13
Data of Purity in FeSSIF Filtrates at 0.5-8 Hours (duplicate samples)

| | HPLC Purity (area %, 220 nm) | | | | | | | | | |
|---|---|---|---|---|---|---|---|---|---|---|
| | Lot# 3, 99.30% | | | | | Lot# 2, 99.28% | | | | |
| | | Sample 1 | | | | | Sample 1 | | | |
| Time | Sample 1/2 | RRT | RRT | RRT | RRT | Sample 1/2 | RRT | RRT | RRT | RRT |
| (hr) | API | 1.09 | 1.74 | 1.91 | 1.95 | API | 1.09 | 1.74 | 1.91 | 1.95 |
| 0.5 | 92.74/92.57 | ND | 0.27 | 0.72 | 0.24 | 96.72/96.89 | ND | 0.33 | 1.00 | 0.32 |
| 2 | 91.52/90.33 | 0.22 | 0.35 | 1.09 | 0.39 | 80.67/77.22 | 1.18 | 1.52 | 5.63 | 0.89 |
| 4 | 84.44/77.77 | 1.00 | 0.82 | 3.55 | 0.69 | 65.06/60.63 | 3.56 | 2.64 | 9.12 | 1.67 |
| 8 | 66.91/57.55 | 4.10 | 2.41 | 7.91 | 1.57 | 50.99/47.77 | 6.24 | 5.16 | 8.77 | 3.62 |

### Suspension Formulation Stability in 0.5% MC pH 3 Buffer

Solubility of freebase Form II was first measured in 0.5% MC (400 cPs) and 0.5% MC (400 cPs) pH 3 citrate buffer. About 5 mg of Form II (Lot#1) was stirred in 1 mL of media at RT for ˜2 hours. The suspension was filtered, with filtrates analyzed by HPLC and remaining solids tested by XRPD. The results are summarized in Table 14. Freebase Form II showed a high solubility of 4.5 mg/mL in 0.5% MC pH 3 citrate buffer, but limited solubility <10 μg/mL in 0.5% MC. No form change was observed in both media. Therefore, 0.5% MC pH 3 buffer was selected for suspension formulation stability study.

About 9 mg of free base Form II (Lot#1) was added into 0.3 mL of 0.5% MC pH 3 buffer, and stirred at RT for 30 min to make a suspension with 30 mg/mL drug loading (Day 0 sample). The suspension samples in vials, prepared at the same time, were kept at RT for 3 or 7 days. At each time point, the whole suspension in a vial was dissolved in 8.7 mL water/ACN (1:1, v/v) and then was analyzed by HPLC. Sedimentation of suspension was checked by visual observation, and the solids in another vial were tested by PLM and XRPD.

The results are summarized in Table 15. Sedimentation was observed after being kept at RT for 3 or 7 days, and homogeneous suspension could be achieved after swirled at RT for 1 min. Fine crystals (<5 μm) and small aggregates were observed in the formulation under microscope. Freebase Form II remained unchanged in 0.5% MC pH 3 citrate buffer for up to 7 days, and no purity decrease was observed.

TABLE 14
Data of Solubility of Form II in Media

| | | | | Filtrate |
|---|---|---|---|---|
| | Initial | Solubility | | Purity |
| Medium | pH | (mg/mL) | XRPD | 254 nm |
| 0.5% MC pH 3 buffer | 3.0 | 4.5 | Unchanged | 99.25% |
| (100 mM citrate) | | | | |
| 0.5% MC | N/A | 0.006 | | — |

TABLE 15
Data of Suspension Formulation Stability

| | Drug | | | | |
|---|---|---|---|---|---|
| Suspension Formulation | Loading | Purity (area %, 220 nm) | | | |
| Sample (Lot#) | (mg/mL) | Initial | Day 0 | Day 3 | Day 7 |
| 0.5% MC pH 3 buffer | 30 | 99.25 | 99.30 | 99.28 | 99.26 |
| (100 mM citrate) | | | | | |
| Freebase Form II | | | | | |
| (A14259-052A1-DVS) | | | | | |

### Solid-State Stability

Solid-state stability of free base Form II (Lot#1) was conducted at 60° C. (capped) and 40° C./75% RH (open) for 7 days. The stability sample was dissolved in diluent to prepare solution at ˜0.5 mg/mL for HPLC purity analysis. Solid samples were analyzed by XRPD to check the crystalline form. The results are summarized in Table 16. Freebase Form II was physically and chemically stable at both 60° C. (capped) and 40° C./75% RH (open) after 7 days.

TABLE 16
Stability Evaluation Results of Form II

| Purity (area %) @7 D | | | XRPD @7 D | |
|---|---|---|---|---|
| Initial | 40° C./75% RH | 60° C. | 40° C./75% RH | 60° C. |
| 99.26 | 99.26 | 99.25 | Unchanged | Unchanged |

### Slurry Conversion

Appropriate amount of starting material was added into different solvents to make suspensions, which were kept stirring at RT for 7 days or at 50° C. for 4 days. Solid samples were collected by centrifugation and analyzed by XRPD. Freebase Form II were obtained by slurry in most solvent. The results are summarized in Table 17 and Table 18.

TABLE 17
Results of Slurry at RT

| No. | Solvent (Vol.) | XRPD @Day 7, Wet |
|---|---|---|
| 1 | MeOH (40 V) | Form II |
| 2 | Acetone (40 V) | Form II |
| 3 | ACN (40 V) | Form II + I |
| | | (trace) |
| 4 | THF (40 V) | Form II |
| 5 | DCM (40 V) | Form II |
| 6 | Toluene (40 V) | Form I + II |
| 7 | EtOAc (40 V) | Form I + II |
| 8 | n-Heptane (40 V) | Form I + II |
| 9 | Water (40 V) | Form II |
| 10 | Anisole (40 V) | Form I + II |

TABLE 18
Results of Slurry at 50° C.

XRPD @Day 4

| No. | Solvent (Vol.) | Wet | Dry |
|---|---|---|---|
| 1 | MeOH (32 V) | Form II | Form II |
| 2 | Acetone (32 V) | Form II | Form II |
| 3 | ACN (32 V) | Form II | Form II |
| 4 | THF (32 V) | Form II | Form II |
| 5 | MIBK (32 V) | Form I + II | Form I + II |
| 6 | Toluene (32 V) | Form I + II | Form I + II |
| 7 | EtOAc (32 V) | Form II | Form II + I |
| | | | (trace) |
| 8 | n-Heptane (32 V) | Form I + II | Form I + II |
| 9 | Water (32 V) | Form II | Form II |
| 10 | Anisole (32 V) | Form I + II | Form I + II |

### Quench Cooling

Quench cooling was performed in 4 selected solvents. About 20 mg of starting material was weighed into a glass vial, and then selected solvent was added to make a suspension with stirring at 50° C. Then the suspension was filtered to obtain saturated drug solution, which was cooled to 5° C. directly. Any solid obtained was characterized by XRPD. The results are summarized in Table 19. Form II was obtained in MeOH and acetone.

TABLE 19
Results of Quench Cooling

| No. | Solvent (Vol.) | Result |
|---|---|---|
| 1 | DCM (500 V) | Form I (little |
| | | solid) |
| 2 | THF (500 V) | Trace solid |
| 3 | MeOH (400 V) | Form II |
| 4 | Acetone (1000 V) | Form II |

### Anti-Solvent Precipitation

Anti-solvent precipitation was performed by adding anti-solvent dropwise to the prepared drug solution at RT. Appropriate amount of starting material was weighed into glass vials, and then selected solvent was added to make nearly saturated solution. After filtration, anti-solvent was added into the filtrate gradually until solids precipitated out or 7V anti-solvent was added at RT. If precipitation occurred, solids were isolated by centrifugation and characterized accordingly. The results are summarized in Table 20. Pattern III was obtained in DMSO/water, and pure Form I was obtained in DCM/MeOH with MTBE as anti-solvent.

TABLE 20
Results of Anti-Solvent Precipitation

| | Anti- | Solvent (v:v) | | Result | |
|---|---|---|---|---|---|
| No. | solvent | Vol. | V₁/V₂ | ~30 Min | 1 Day |
| 1 | Acetone | DMSO, 60 V | 1/7 | Solution | Form II (7 d) |
| 2 | ACN | | 1/7 | Form II | Form II |
| 3 | DCM | | 1/7 | Solution | Solution |
| 4 | EtOH | | 1/3 | Solution | Solution |
| 5 | THF | | 1/2 | Solution | Solution |
| 6 | Water | | 1/2 | Pattern | Pattern III (wet) |
| | | | | III | Form II (dry) |
| 7 | n-Heptane | MeOH/DCM | 1/2 | Form II | Form II |
| 8 | ACN | (1:1), 30 V | 1/2 | Form II | Form II |
| 9 | EA | | 1/4 | Form II | Form II |
| 10 | MTBE | | 1/4 | Form I | Form I |
| 11 | MTBE | | 1/4 | Form I | N/A |

V₁/V₂ is volume ratio of solvent to anti-solvent.

### Slow Evaporation

Evaporation was performed in 3 selected solvents according to the solubility data. A clear solution was prepared in a glass vial. Then the vial was covered with pin-hole film and placed at RT for slow evaporation until solid precipitation. The results are summarized in Table 21. Form II was obtained in DCM and MeOH.

TABLE 21
Results of Slow Evaporation

| No. | Solvent (Vol.) | Result |
|---|---|---|
| 1 | DCM (800 V) | Form II |
| 2 | THF (800 V) | Form I + II |
| | | (trace) |
| 3 | MeOH (800 V) | Form II |

### Analysis Methods

### PLM

Light microscopy analysis was performed using an ECLIPSE LV100POL (Nikon, JPN) microscope. Each sample was placed on a glass slide with a drop of immersion oil and covered with a glass slip. The sample was observed using a 4-20× objective with polarized light.

### XRPD

XRPD diffractograms were collected with an X-ray diffractometer. The sample was prepared on a zero-background silicon wafer by gently pressing onto the flat surface. The parameters of XRPD diffraction are given in the table below.

Parameters for XRPD Testing

| | |
|---|---|
| Instrument | PANalytical, Empyrean |
| Radiation | Cu Kα (λ = 1.5418 Å) |
| Detector | PIXcel^{1D} |
| Scan angle | 3-40° (2θ) |
| Scan step | 0.013° (2θ) |
| Scan speed | 20.4 s/step |
| Tube voltage/current | 45 kV/40 mA |
| Divergence slit | ⅛° |
| Rotation | On |
| Sample holder | Zero-background sample pan |

### TGA

TGA analysis was performed using a TA Instrument. About 1-5 mg of a sample was loaded onto a pre-tared aluminum pan and heated with the parameters in the table below. The data was analyzed using TRIOS.

Parameters of TGA Testing

| | |
|---|---|
| Instrument | TA, Discovery TGA 55 |
| Sample pan | Aluminum, open |
| Temperature range | RT-300° C. |
| Heating rate | 10° C./min |
| Purge gas | N₂ |
| Flow rate | Balance chamber: 40 mL/min |
| | Sample chamber: 60 mL/min |

### DSC

DSC analysis was performed with a TA Instrument. About 1-3 mg of a sample was placed into an aluminum pan with pin-hole and heated with the parameters in the table below. The data was analyzed using TRIOS.

Parameters of DSC Analysis

| | |
|---|---|
| Instrument | TA, Discovery DSC 250 |
| Sample pan | Aluminum, pin-holed |
| Temperature range | 25-300° C. |
| Heating rate | 10° C./min |
| Purge gas | N₂ |
| Flow rate | 50 mL/min |

### DVS

Moisture sorption/desorption data were collected on a DVS instrument. About 75 mg of a sample was placed into a tared sample chamber and automatically weighed. The anhydrate was analyzed with the setting parameters in the table below.

The Parameters of DVS Analysis for Anhydrates

| Instrument | ProUmid GmbH & Co. KG | |
|---|---|---|
| Sample temperature | 25° C. | |
| Time between cycles | 10 min | |
| Minimum time per climate setting | 50 min | |
| Maximum time per climate setting | 120 min | |
| Weight limit | 100% | |
| Equilibrium condition | 0.01%/45 min | |
| Climate cycle #1 | 0%-0% | 1 step at 40° C. for 3 h |
| Climate cycle #2 | 0%-90% | 9 steps at 25° C. |
| Climate cycle #3 | 80%-0% | 8 steps at 25° C. |

| | |
|---|---|
| Adsorption | 0, 10, 20, 30, 40, 50, 60, 70, 80, 90 |
| Desorption | 80, 70, 60, 50, 40, 30, 20, 10, 0 |
| desorption | 0%-0% |

### ¹H-NMR

¹H-NMR spectra were collected on a Bruker 400 MHz instrument. Unless specified, samples were prepared in DMSO-d₆ solvent and measured with the parameters in the table below. The data was analyzed using MestReNova.

Parameters for ¹H-NMR Analysis

| Instrument | Bruker | |
|---|---|---|
| Frequency | 400 | mHz |
| Scan times | 4 | |
| Temperature | 295 | K |
| Relaxation delay | 1 | s |

### HPLC

HPLC analysis was performed with an Agilent HPLC 1260 series instrument. HPLC method for solubility and stability testing is presented in the table below.

HPLC Method for Solubility and Stability Testing

| | |
|---|---|
| Instrument | Agilent 1260 HPLC series |
| Column | Ascentis Express C18, 4.6 × 100 mm, 2.7 μm |
| Mobile Phase | A: 0.05% TFA in water; B: 0.05% TFA in ACN |
| Gradient (T/B %) | 0.0/5%, 7/20%, 8/20%, 13/35%, 16/95%, 17/95%, 17.1/5% |
| Column Temperature | 30° C. |
| Detector | DAD; 220 nm |
| Flow Rate | 1 mL/min |
| Injection Volume | 2 or 3 μL |
| Run Time | 17.10 min |
| Post Time | 3 min |
| Diluent | MeOH/water (v/v, 9:1) |

1. A crystalline form of 5-(4-((7-cyclopropyl-6-oxo-5,6-dihydro-1,5-naphthyridin-3-yl)methyl)piperazin-1-yl)-N-methylpicolinamide (compound 1): 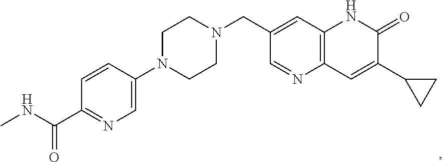 [SMILES: CNC(=O)c1ccc(N2CCN(Cc3cnc4cc(C5CC5)c(=O)[nH]c4c3)CC2)cn1]
or a pharmaceutically acceptable salt or solvate thereof.

2. A crystalline form of freebase 5-(4-((7-cyclopropyl-6-oxo-5,6-dihydro-1,5-naphthyridin-3-yl)methyl)piperazin-1-yl)-N-methylpicolinamide (compound 1): 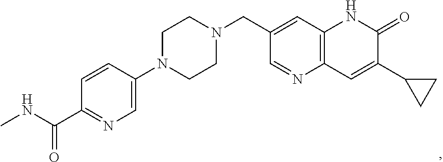 [SMILES: CNC(=O)c1ccc(N2CCN(Cc3cnc4cc(C5CC5)c(=O)[nH]c4c3)CC2)cn1]
or a pharmaceutically acceptable solvate thereof.

3. The crystalline form of claim 2, wherein the crystalline form is freebase 5-(4-((7-cyclopropyl-6-oxo-5,6-dihydro-1,5-naphthyridin-3-yl)methyl)piperazin-1-yl)-N-methylpicolinamide (compound 1):

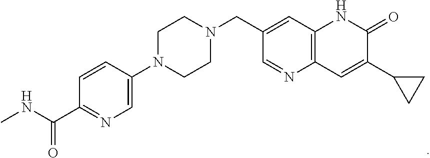 [SMILES: CNC(=O)c1ccc(N2CCN(Cc3cnc4cc(C5CC5)c(=O)[nH]c4c3)CC2)cn1]

4. (canceled)

5. (canceled)

6. The crystalline form of claim 1, wherein the crystalline compound 1 is Form II characterized as having at least one of the following properties:
(a) an X-ray powder diffraction (XRPD) pattern substantially the same as shown in FIG. 2;
(b) an X-ray powder diffraction (XRPD) pattern with characteristic peaks at 20.88±0.2° 2θ, 21.37±0.2° 2θ, and ±25.31 0.2° 2θ,
(c) a DSC thermogram with an endotherm having an onset temperature at about 265.5° C. and a peak temperature at about 266.5° C.; or
(d) combinations thereof.

7. The crystalline form of claim 6, wherein the crystalline form has an X-ray powder diffraction (XRPD) pattern substantially the same as shown in FIG. 2.

8. (canceled)

9. The crystalline form of claim 6, wherein crystalline compound 1, Form II is characterized by an X-ray powder diffraction (XRPD) pattern with characteristic peaks at 20.88±0.2° 2θ, 21.37±0.2° 2θ, and ±25.31 0.2° 2θ.

10. The crystalline form of claim 9, wherein the X-ray powder diffraction pattern further comprises a peak at 10.96±0.2° 2θ.

11. The crystalline form of claim 10, wherein the X-ray powder diffraction pattern further comprises a peak at 16.57±0.2° 2θ.

12. The crystalline form of claim 11, wherein the X-ray powder diffraction pattern further comprises a peak at 17.26±0.2° 2θ.

13. The crystalline form of claim 12, wherein the X-ray powder diffraction pattern further comprises a peak at 17.71±0.2° 2θ.

14. The crystalline form of claim 13, wherein the X-ray powder diffraction pattern further comprises a peak at 17.93±0.2° 2θ.

15. The crystalline form of claim 14, wherein the X-ray powder diffraction pattern further comprises a peak at 19.54±0.2° 2θ.

16. The crystalline form of claim 15, wherein the X-ray powder diffraction pattern further comprises a peak at 23.25±0.2° 2θ.

17. The crystalline form of claim 15, wherein the X-ray powder diffraction pattern further comprises a peak at 26.27±0.2° 2θ.

18.-22. (canceled)

23. The crystalline form of claim 6, wherein crystalline compound 1, Form II is characterized by a DSC thermogram with an endotherm having an onset temperature at about 265.5° C. and a peak temperature at about 266.5° C.

24. The crystalline form of claim 6, wherein crystalline compound 1, Form II is anhydrous.

25. (canceled)

26. (canceled)

27. The crystalline form of claim 1, wherein the crystalline compound 1 is Form I characterized as having at least one of the following properties:
(a) an X-ray powder diffraction (XRPD) pattern substantially the same as shown in FIG. 1;
(b) an X-ray powder diffraction (XRPD) pattern with characteristic peaks at 8.11±0.2° 2θ, 16.80±0.2° 2θ, 19.35±0.2° 2θ, 20.44±0.2° 2θ, and 24.46±0.2° 2θ.
(c) a DSC thermogram with an endotherm having a peak temperature at about 255° C.;
(d) a DSC thermogram with an endotherm having a peak temperature at about 258° C.;
(e) a DSC thermogram with an endotherm having a peak temperature at about 266° C.;
(f) a thermogravimetric analysis (TGA) thermogram comprising a loss in mass of about 1.4% over a temperature range of about 70° C. to about 255° C.; or
(g) combinations thereof.

28. The crystalline form of claim 27, wherein crystalline compound 1, Form I is characterized by an X-ray powder diffraction (XRPD) pattern substantially the same as shown in FIG. 1.

29. (canceled)

30. The crystalline form of claim 27, wherein crystalline compound 1, Form I is characterized by an X-ray powder diffraction (XRPD) pattern with characteristic peaks at 8.11±0.2° 2θ, 16.80±0.2° 2θ, 19.35±0.2° 2θ, 20.44±0.2° 2θ, and 24.46±0.2° 2θ.

31. (canceled)

32. (canceled)

33. The crystalline form of claim 30, wherein the X-ray powder diffraction pattern further comprises at least one peak selected from 10.10±0.2° 2θ, 12.14±0.2° 2θ, 22.25±0.2° 2θ, and 25.22±0.2° 2θ.

34.-42. (canceled)

43. The crystalline form of claim 27, wherein crystalline compound 1, Form I is characterized by a DSC thermogram with an endotherm having a peak temperature at about 255° C.; or a DSC thermogram with an endotherm having a peak temperature at about 258° C.; or a DSC thermogram with an endotherm having a peak temperature at about 266° C.

44. (canceled)

45. (canceled)

46. The crystalline form of claim 27, wherein crystalline compound 1, Form I is characterized by a thermogravimetric analysis (TGA) thermogram comprising a loss in mass of about 1.4% over a temperature range of about 70° C. to about 255° C.

47. The crystalline form of claim 27, wherein crystalline compound 1, Form I is anhydrous.

48. A pharmaceutical composition comprising a crystalline form of claim 1, and a pharmaceutically acceptable excipient.

49-52. (canceled)

53. A method of treating cancer in a subject in need thereof, the method comprising administering a crystalline form of claim 1.

54. The method of claim 53, wherein the cancer is breast cancer, ovarian cancer, pancreatic cancer, prostate cancer, a hematological cancer, gastrointestinal cancer, or lung cancer.

55. A method of treating a cancer comprising a BRCA1 and/or a BRCA2 mutation in a subject in need thereof, the method comprising administering a crystalline form of claim 1.

56. The method of claim 55, wherein the cancer is bladder cancer, brain & CNS cancers, breast cancer, cervical cancer, colorectal cancer, esophagus cancer, Hodgkin lymphoma, non-Hodgkin lymphoma, kidney cancer, leukemia, lung cancer, melanoma, myeloma, oral cavity cancer, ovarian cancer, pancreatic cancer, prostate cancer, skin cancer, stomach cancer, thyroid cancer, or uterus cancer.
